(19) United States
(12) Patent Application Publication
(10) Pub. No.: US 2022/0120761 A1
(43) Pub. Date: Apr. 21, 2022

(54) Urine metabolomics based method of detecting renal allograft injury

(71) Applicant: The Regents of the University of California, Oakland, CA (US)

(72) Inventors: Minnie Sarwal, San Francisco, CA (US); Joshua Yang, San Francisco, CA (US)

(73) Assignee: The Regents of the University of California, Oakland, CA (US)

(21) Appl. No.: 17/423,868

(22) PCT Filed: Jan. 17, 2020
(86) PCT No.: PCT/US2020/014093

§ 371 Date: Jul. 17, 2021

Related U.S. Application Data
(60) Provisional application No. 62/793,870, filed on Jan. 17, 2019.

(51) Int. Cl. G01N 33/68; G16H 50/30; G16B 40/20; A61K 45/06

(52) U.S. Cl. CPC G01N 33/6893; G16H 50/30; G16B 40/20; G01N 2800/52; G01N 33/6848; G01N 2800/245; G01N 2800/347; A61K 45/06

(57) ABSTRACT

The disclosure describes a comprehensive metabolome analysis of urine samples that identified panels of metabolite markers for diagnosis and monitoring of alloimmune injury, acute rejection, and BK virus nephropathy. The disclosure provides non-invasive ways to monitor the status of transplanted kidneys by monitoring the presence of defined metabolite panels over a period of time. The metabolite panels of the disclosure can distinguish the between kidney injuries of distinct etiology with high sensitivity and specificity.

### CROSS-REFERENCE

This application is a 35 USC § 371 national stage application of International Patent Application Number PCT/US2020/014093, entitled “Urine metabolomics based method of detecting renal allograft injury,” filed Jan. 17, 2020, which claims the benefit of priority to U.S. Provisional Application No. 62/793,870, entitled “Urine metabolomics based method of detecting renal allograft injury,” filed on Jan. 17, 2019, which application is incorporated herein by reference.

### STATEMENT AS TO FEDERALLY SPONSORED RESEARCH

This invention was made with government support under grant no. R01 DK083447 awarded by the National Institutes of Health. The government has certain rights in the invention.

### BACKGROUND

For subjects with end-stage renal disease, kidney transplantation is the gold-standard mode of therapy. However, despite advancements in surgical tools and procedures, improved organ procurement, and immunosuppressive drugs, potential immune and non-immune related injuries still cause deterioration, dysfunction, and eventual failure of the allograft. Interstitial fibrosis and tubular atrophy (IFTA) remain a primary cause of progressive chronic histological damage and graft loss 5 years post-transplantation. Longevity of transplanted kidneys is critical because of the shortage of available kidneys and kidney donors. There is a need for better means for detecting and analyzing biomarkers present in the body of subjects having or at risk of developing various diseases or disorders, particularly in the context of organ transplantation. The present invention addresses this and other needs.

### SUMMARY

Kidney transplant is the gold standard treatment in end-stage renal disease with kidney failure with over 100,000 people in the United States on the transplant waiting list every year. Despite the high need for organs, less than 20,000 people a year in the United States will receive a kidney transplant off the wait list. Due to this shortage, organ longevity is a critical concern and transplant recipients undergo lifelong monitoring of the organ for disease, dysfunction, and rejection. Unfortunately, current methods of transplanted organ monitoring are not adequately sensitive and specific and definitive diagnosis of common allograft injuries requires kidney biopsy, a costly and morbid procedure that can only be used in limited clinical scope. While this therapeutic approach has become a routine practice worldwide, significantly improving subject quality of life and survival, long-term kidney allograft outcomes have not improved. Despite a better understanding of the immune biology of allograft rejection and the advent of novel and more potent immunosuppressive agents the standard-of-care approaches have not provided an increase in longevity of the transplant. Thus, effective noninvasive ways to monitor the status of transplanted kidneys remains an unmet need.

In one aspect, the present disclosure encompasses novel methods of detecting kidney allograft injury in a transplant recipient. The methods of the invention are based on the discovery of biomarker profiles that are indicative of various forms of kidney allograft injury, including acute rejection (AR), BK virus nephropathy (BKVN), and chronic allograft nephropathy (CAN).

In some aspects the disclosure provides a method of distinguishing a stable kidney allograft from a kidney allograft afflicted by an alloimmune injury comprising: (a) obtaining a sample from a subject that received a kidney allograft; (b) detecting a panel of metabolites in the sample of the subject; and (c) distinguishing if the kidney allograft is stable or is afflicted by an alloimmune injury by inputting data from the detection of the panel of metabolites into a predictive model, wherein the output of the model is indicative of allograft status. The panel of metabolites may include a combination of various types of metabolites, including a combination of amino acids, amino acid derivatives, carbohydrates, organic molecules, and other compounds. The sample may be a urine sample. The method of detection may be mass spectroscopy analysis.

In some cases, the alloimmune injury is acute rejection. In such instances the panel of metabolites may comprise at least 3 metabolites, for example, a 3-metabolite panel, in some embodiments being a panel including glycine, N-methylalanine, and inulobiose. In other cases, the alloimmune injury may be detected by a 4-metabolite panel, a 5-metabolite panel, a 6-metabolite panel, a 7-metabolite panel, an 8-metabolite panel, a 9-metabolite panel, or a 11-metabolite panel including glycine, N-methylalanine, and inulobiose and one or more of adipic acid, glutaric acid, threitol, isothreitol, sorbitol, and isothreonic acid. In some embodiments, the panel for differentiating AR from stable subjects may comprise at least 11 metabolites, for example, an 11-metabolite panel, wherein the panel includes glycine, glutaric acid, adipic acid, inulobiose, threose, sulfuric acid, taurine, N-methylalanine, asparagine, 5-aminovaleric acid lactame, and myoinositol. When the 11-metabolite AR panel is selected, it may have a sensitivity greater than 80%, 85%, or 90%, and a specificity greater than 80%, 85%, or 90%, for detecting the acute rejection. When the 11-metabolite AR panel is selected, it may include a combination of at least one amino acids, at least one amino acid derivative, at least one carbohydrate, and at least one organic compound. In various embodiments, the panel comprises a subset of two, three, four, five, six, seven, eight, nine or ten metabolites selected from the group consisting of glycine, glutaric acid, adipic acid, inulobiose, threose, sulfuric acid, taurine, N-methylalanine, asparagine, 5-aminovaleric acid lactame, and myoinositol. For instance, Glycine (symbol Gly or G) is an amino acid that has a single hydrogen atom as its side chain. It is the simplest amino acid (since carbamic acid is unstable), with the chemical formula NH₂—CH₂—COOH. N-Methylalanine, also known as (S)-2-methylaminopropanoate or N-methyl-L-alanine, is classified as an alanine or an alanine derivative. Adipic acid or hexanedioic acid is an organic compound with the formula (CH₂)₄(COOH)₂. Glutaric acid is the organic compound with the formula C₃H₆(COOH)₂. Inulobiose (1-β-d-fructofuranosylfructose) is carbohydrate. Threitol is a carbohydrate, specifically a four-carbon sugar alcohol, with the molecular formula C₄H₁₀O₄. Sorbitol is a carbohydrate, specifically it is a sugar alcohol. Threonic acid is a sugar acid derived from threose. Inositol, or more precisely myo-inositol, is a carbocyclic sugar.

In some cases, the alloimmune injury is chronic allograft nephropathy (CAN). In such instances the panel of metabolites may be a panel comprising at least 9 metabolites, for example, a 9-metabolite panel, wherein the panel comprising at least 9 metabolites includes glycine, N-methylalanine, adipic acid, glutaric acid, inulobiose, threitol, isothreitol, sorbitol, and isothreonic acid. When the 9-metabolite CAN panel is selected, it may have a sensitivity greater than 80%, 85%, 90%, or 95%, and a specificity greater than 60%, 65%, 70%, or 75%, for detecting the chronic allograft nephropathy. When the 9-metabolite CAN panel is selected, it may include a combination of at least one amino acids, at least one amino acid derivative, at least one mineral, at least one carbohydrate, and at least one organic compound. In various embodiments, the panel for differentiating CAN from stable grafts comprises one, two, three, four, five, six, seven, or eight metabolites selected from the group consisting of glycine, N-methylalanine, adipic acid, glutaric acid, inulobiose, threitol, isothreitol, sorbitol, and isothreonic acid. N-methylalanine, glycine, adipic acid, glutaric acid, and inulobiose have been described above. Sulfuric acid, also known as vitriol, is a mineral acid composed of the elements sulfur, oxygen and hydrogen, with molecular formula H₂SO₄.

Taurine, or 2-aminoethanesulfonic acid, is an organic compound, specifically an amino sulfonic acid, but it is often referred to as an amino acid for its importance as a building block. Threose is a carbohydrate, specifically a four-carbon monosaccharide with molecular formula C4H8O4.

In some cases, the alloimmune injury is BKVN infection. In such instances the panel of metabolites may be a panel comprising at least 5 metabolites, for example, a 5-metabolite panel, wherein the panel comprising at least 5 metabolites includes arabinose, 2-hydroxy-2-methylbutanoic acid, hypoxanthine, benzylalcohol, and N-acetyl-D-mannosamine. When the 5-metabolite BKVN panel is selected, it may have a sensitivity greater than 70%, 75%, 80%, or 85%, and a specificity greater than 80%, 85%, or 90%, for detecting the BKVN infection. When the 5-metabolite BKVN panel is selected, it may include a combination of at least one nucleobase, at least one carbohydrate, at least one fatty acid, and at least one organic compound. In various embodiments, the panel for differentiating BKNV from stable graft status comprises one, two, three, or four metabolites selected from the group consisting of arabinose, 2-hydroxy-2-methylbutanoic acid, hypoxanthine, benzylalcohol, and N-acetyl-D-mannosamine. Arabinose is a carbohydrate, specifically an aldopentose—a monosaccharide containing five carbon atoms, and including an aldehyde functional group. 2-hydroxy-2-methylbutyric acid is a branched-chain fatty acid. Hypoxanthine is a nucleobase, specifically a purine nucleobase that consists of purine bearing an oxo substituent at position 6. Benzylalcohol is an organic compound, specifically an aromatic alcohol with the formula C₆H₅CH₂OH. N-Acetylmannosamine is a carbohydrate, specifically a hexosamine monosaccharide.

In some instances, the mass spectroscopy analysis is a gas chromatography—mass spectrometry (GC-MS) analysis, a capillary electrophoresis—mass spectrometry (CE-MS) analysis, a liquid chromatography—mass spectrometry (LC-MS) analysis. In some case, the methods further comprises monitoring a status of the kidney allograft by repeating steps (a) through (c) over a period-of-time. The period-of-time may be within a week, within a month, within 6-months, within 1-year or within another suitable time of the subject receiving the kidney allograft. In some cases, the stability of the urine sample needs to be preserved by refrigeration of the urine sample after sample collection. In some cases, the urine sample of the subject is obtained no more than a week or no more than a month prior to performing step (b). In some instances, the predictive model is based on a nonlinear, nonparametric machine learning analysis of the metabolite data. For instance, the predictive model can be based on a variable selection method based on a random forests model of the metabolite data, and in such cases the variable selection method can be based on the VSURF random forests model. In some instances, the predictive model is based on symbolic regression.

In some cases, the method further comprises a step of administering a drug for treating the diagnosed disorder. In some cases, the treatment comprises administering an effective amount of an immunosuppressive drug when the alloimmune injury is detected. In some cases, the immunosuppressive drug is a calcineurin inhibitor, such as cyclosporin. In other cases, the immunosuppressive drug is belatacept. In some instances, the immunosuppressive drug is a lymphocyte depleting antibody, such as Thymoglobulin. In other instances, the immunosuppressive drug is mycophenolate or azathioprine. In some instances the drug is a corticosteroid. Yet in other instances, the method further comprises administering an effective amount of an intravenous immunoglobulin when the alloimmune injury is detected.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 (FIG. 1) is a schematic of a study in human subjects. The schematic outlines the flow of study samples, assay platform, study phenotypes, analysis, and results.

FIG. 2 (FIG. 2) depicts a heat map of the data used for supervised clustering.

FIG. 3 (FIG. 3) depicts a z-score plot of the date used for supervised learning.

FIG. 4 (FIG. 4) depicts the identification of a potential biomarker panel of metabolites for transplant alloimmune injury and acute rejection using VSURF method (Performance of Random Forests (RF) prediction model). FIG. 2 depicts three beanplots demonstrating distribution of 3 most significant metabolites in Acute Rejection (AR) compared to stable kidney grafts with normal protocol biopsies (STA), namely glycine, N-methylalanine, inulobiose. The bold horizontal line represents mean value for each group.

FIG. 5 (FIG. 5) depicts an ROC curve representing prediction accuracies and a statistical comparison of the full and sparse RF models for alloimmune injury and the table displaying classification accuracy on test set. Out of 266 metabolites tested in the model, a 9-metabolite panel including glycine, N-methylalanine, adipic acid, glutaric acid, inulobiose, threitol, isothreitol, sorbitol, and isothreonic acid could differentiate alloimmune injury with greater than 95% sensitivity.

FIG. 6 (FIG. 6) depicts an ROC curve representing prediction accuracies and a statistical comparison of the full and sparse RF models for alloimmune injury and the table displaying classification accuracy on test set. Out of 266 metabolites tested in the model, a 11-metabolite panel including glycine, glutaric acid, adipic acid, inulobiose, threose, sulfuric acid, taurine, N-methylalanine, asparagine, 5-aminovaleric acid lactame, and myoinositol could differentiate AR injury with greater than 95% sensitivity.

FIG. 7 (FIG. 7). shows volcano plot displaying fold change and significance of metabolites. Red dots denotes metabolites significant at the Bonferroni-adjusted level. The right half displays metabolites in the injury group with a higher signature relative to the stable group. Some metabolites from 9-metabolite marker panel for alloimmune injury and 11-metabolite marker panel for AR are among the very highly perturbed metabolites.

FIG. 8 (FIG. 8) is a graph illustrating enrichment analysis of metabolic pathways using significantly altered metabolites showed enrichment in nitrogen metabolism (p=0.0055), ascorbate and aldarate metabolism (p=0.0083), and amino sugar and nucleotide sugar metabolism (p=0.05) as significantly enriched pathways.

FIG. 9 (FIG. 9) depicts metabolic pathways impacted by Belatacept.

FIG. 10 (FIG. 10) depicts metabolic pathways impacted by Cyclosporin.

FIG. 11 (FIG. 11) depicts enrichment in metabolic activities based on the urine metabolites specific to either the Belatacept or the Cyclosporin treatment arms.

FIG. 12 (FIG. 12) depicts an integrative analysis of gene expression level of myo-inositol transporting gene SMIT provides a clue why myo-inositol is increased in AR urine.

### DETAILED DESCRIPTION

Kidney transplantation is the preferred method of treatment for end-stage kidney failures. Longevity of transplanted kidneys is critical because of the shortage of available kidneys and kidney donors. However, methods currently used for diagnosing allograft injury and monitoring transplanted organs are not adequately sensitive or specific. A major cause for persistent and poor graft survival is the inability to non-invasively quantify the burden of graft immune injury and predict Acute Rejection (AR) prior to substantive functional decline and histological injury. Indeed, while it is well known that kidney transplant subjects are continuously exposed to immune and nonimmune related injuries, periodic kidney transplant monitoring is dependent on insensitive surrogate markers of allograft dysfunction—such as serum creatinine. Additionally, sporadic monitoring is based on invasive protocol allograft biopsies to detect sub-clinical histological graft injury in the absence of perturbation of the serum creatinine.

Though assessment of graft dysfunction based only on the serum creatinine has sensitivity for non-specific, established, allograft damage, it has low specificity for distinguishing defined allograft injuries, such as acute rejection (AR). Often, a rise in the serum creatinine of an allograft recipient can be due to other reasons not directly related to allograft rejection, such as immunosuppressive (IS) drug-related nephrotoxicity, acute tubular necrosis, infection, and interstitial fibrosis and tubular atrophy (IFTA). Furthermore, while the use of surveillance biopsies has been postulated as the gold standard tool for diagnosing allograft lesions, this approach is costly, invasive, with procedure morbidity (risk of bleeding; procedure requiring sedation, particularly for pediatric kidney transplant subjects), fraught with inter-operator read variabilities, and is often poorly representative of focal histological injury. Therefore, the use of non-invasive biological markers that can accurately predict and quantify the burden of immune injury in the allograft would be a significant advance for precision kidney transplant monitoring.

Several efforts have been made to identifying surrogate biomarkers for kidney transplant dysfunction using gene expression analyses and proteomics methods. However, previous efforts to evaluate urine biomarkers as a non-invasive diagnostic approach for the analysis kidney transplantation have focused almost exclusively on single biomarkers, including chemokines and receptors such as CXCR3, CXCL9, or CXCL10. Such efforts failed to capture the molecular complexity and heterogeneity of transplant rejections, particularly of acute rejection (AR) across a population of kidney transplant subjects. Capturing this heterogeneity is essential to quantify the burden of injury in a manner usable for prospective monitoring of AR and recovery of graft injury after therapeutic intervention.

Disclosed herein are methods for distinguishing various kidney and allograft diseases based on a panel of biomarkers. Acute rejection (AR) episodes are a major cause of renal allograft failure. Chronic allograft nephropathy (CAN), which is characterized with chronic interstitial fibrosis and tubular atrophy within the renal allograft, is the leading cause of allograft loss in pediatric renal transplant recipients. CAN is characterized by a gradual decline in kidney function, despite the use of immunosuppressive regimens. BK virus is a type of polyomavirus. Although the virus is latent and asymptomatic in most situations, in kidney recipients undergoing immunosuppression treatment, the virus can reactivate, endangering graft survival. Disclosed herein are panels of biomarkers that can be used to distinguish common allograft injuries such as acute rejection (AR), chronic allograft nephropathy (CAN), and BK virus nephropathy from normal allografts. This work establishes a noninvasive yet sensitive and accurate method for diagnosis of renal allograft disfunction that aids in the monitoring of kidney transplants.

### Definitions

As used herein, the term “allograft” refers to the transplant of an organ or tissue from one individual to another of the same species but with a different genotype. Allografts make up the majority of human organ transplants and may be from living, related, unrelated, or cadaveric donors. An allografted organ may require immune suppressing drugs to prevent rejection.

As used herein, the term “acute rejection” (AR) refers to the rejection of an allografted organ in the days to weeks after transplantation. The immune system sees the grafted organ as foreign and attacks it, destroying it and leading to rejection of the organ shortly after transplantation. The induction of tolerance in alloreactive donor tissue is the major goal in transplantation to prevent rejection and may be managed with immunosuppressive drugs. As shown herein, acute rejection (AR) was defined at minimum, as per Banff Schema, a tubulitis score>1 accompanied with an interstitial inflammation score>1.

As used herein, the term “chronic allograft nephropathy” (CAN) refers to chronic interstitial fibrosis and tubular atrophy commonly seen in kidney transplants. CAN is distinct from chronic rejection (which implies ongoing immunological activity) and appears to be the consequence of cumulative transplant damage from time-dependent immune and nonimmune mechanisms which results in a final, chronic pathway of nephron loss and subsequent fibrotic response. Despite improvements in immunosuppression, it is responsible for most allograft losses and remains an important clinical challenge. As shown herein, chronic allograft nephropathy (CAN) was defined at minimum as a tubular atrophy score>1 accompanied by an interstitial fibrosis score>1.

As used herein, the term “BK virus nephropathy” (BKVN) used interchangeably with the term “BK virus” refers to a common human polyoma virus that typically causes mild symptoms in acute disease before disseminating to the kidneys and urinary tract where it remains latent in the body. The more severe secondary disease is typically associated with kidney transplant subjects where their immunosuppressive regimen allows the latent virus to reactivate where they may develop nephritis which may worsen into graft failure. A definitive BKVN diagnosis is made through allograft biopsy showing viral inclusion bodies often associated with infiltrates and tubulitis that may resemble acute rejection. BKVN has variable presentation and may present with features that range from asymptomatic to those similar to acute rejection or interstitial nephritis. As shown herein, BKVN was defined as positivity of polyomavirus PCR in peripheral blood, together with a positive SV40 stain in the concomitant renal allograft biopsy.

Kidney interstitial fibrosis (IF) can be defined as the accumulation of collagen and related molecules in the interstitium. Tubular atrophy (TA) is defined as loss of specialized transport and metabolic capacity and typically manifested by small tubules with cells with pale cytoplasm or dilated, thin tubules. TA is usually associated with IF (often abbreviated IFTA); but probably has distinct mechanisms related to blood flow, glomerular filtration rate (GFR) or tubular continuity loss.

As used herein, the term “end stage renal disease” which may be used interchangeably with the term “kidney failure” refers to the final, permanent stage of chronic kidney disease where kidney function has declined to the point they can no longer function on their own. Due to renal failure, a subject with end-stage renal disease must receive dialysis or a kidney transplantation to survive.

“Graft injury” in this study was defined as a greater than 20% increase in serum creatinine from its previous steady-state baseline value and an associated biopsy that was pathological.

“Stable kidney grafts with normal protocol biopsies” (STA), also referred to as “Normal allografts”, were defined by an absence of significant injury pathology as defined by Banff schema.

As used herein, the term “end stage renal disease” which may be used interchangeably with the term “kidney failure” refers to the final, permanent stage of chronic kidney disease where kidney function has declined to the point they can no longer function on their own. Due to renal failure, a subject with end-stage renal disease must receive dialysis or a kidney transplantation to survive.

As used herein, the term “metabolomics” refers to the comprehensive analysis of metabolites in a biological specimen which may afford detailed characterization of metabolic phenotypes enabling more precise targets, diagnosis, and treatment. A metabolite may refer to a product of metabolism. A metabolite may be generated by the assembly (e.g., alkylation, phosphorylation, or acylation) or fragmentation (e.g., proteolytic cleavage) of biomolecule. Examples of a metabolite may be a peptide, an amino acid, an amino acid, a carbohydrate (such as a sugar), an organic molecule, a nucleobase, a fatty acid, a mineral, or any derivative thereof.

As used herein, amino acids are the following compounds: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Taurine, for example, is an amino acid derivative.

As used herein, the term “mineral” refers to naturally occurring inorganic compound. Examples of a mineral may be a compound that comprises calcium, phosphorus, potassium, sulfur, sodium, chloride, magnesium, iron, zinc, copper, manganese, iodine, selenium, molybdenum, chromium, fluoride, or any combination thereof.

As used herein, the term “carbohydrate” refers to a biomolecule comprising carbon, hydrogen, and oxygen atoms, usually with a hydrogen-oxygen atom ratio of 2:1. A carbohydrate may be a monosaccharide, such as, for example, arabinose, mannose, threose, inulobiose, or derivatives thereof.

As used herein, the term “metabolic profile” refers to the relative level of at least one metabolite (such as a small molecule) present in a biological sample. A metabolic profile may refer to a metabolic profile for a particular biomolecule or a metabolic profile for a plurality of biomolecules.

As used herein, the term “biomarker” refers to a metabolite or small molecule derived therefrom, that is differentially present (i.e., increased or decreased) in a biological sample (i.e., urine) from a subject or a group of subjects that underwent an organ transplant. A biomarker may also be absent. A biomarker is preferably differentially present at a level that is statistically significant.

As used herein, the term “level” refers to the level of one or more biomarkers means the absolute or relative amount or concentration of the biomarker in the sample.

As used herein, the term “reference profile” refers to the metabolic profile that is indicative of a healthy subject or one or more of a disease state, condition or body disorder. Within the reference profile, there can be reference levels of one or more biomarkers (metabolites or small molecules derived therefrom) that may be an absolute or relative amount or concentration of the one or more biomarkers, a presence or absence of the one or more biomarkers, a range of amount or concentration of the one or more biomarkers, a minimum and/or maximum amount or concentration of the one or more biomarkers, a mean amount or concentration of the one or more biomarkers, and/or a median amount or concentration of the one or more biomarkers.

As used herein, the term “urine panel” refers to a test of the urine where various metabolites may be compared to reference values as a diagnostic.

As used herein, the term “AUC” refers to “area under the curve” or C-statistic, which is examined within the scope of ROC (receiver-operating characteristic) curve analysis. AUC is an indicator that allows representation of the sensitivity and specificity of a test, assay, or method over the entire range of test (or assay) cut points with just a single value. An AUC of an assay is determined from a diagram in which the sensitivity of the assay on the ordinate is plotted against 1-specificity on the abscissa. A higher AUC indicates a higher accuracy of the test; an AUC value of 1 means that all samples have been assigned correctly (specificity and sensitivity of 1), an AUC value of 50% means that the samples have been assigned with guesswork probability and the parameter thus has no significance.

As used herein, the term “statistically significant” means at least about a 95% confidence level, preferably at least about a 97% confidence level, more preferably at least about a 98% confidence level and most preferably at least about a 99% confidence level, as determined using parametric or non-parametric statistics, for example, but not limited to ANOVA or Wilcoxon's rank-sum Test, wherein the latter is expressed as p<0.05 for at least about a 95% confidence level.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

As used herein, the term “each,” when used in reference to a collection of items, is intended to identify an individual item in the collection but does not necessarily refer to every item in the collection unless the context clearly dictates otherwise.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Urine Metabolomics Based Method of Detecting Renal Allograft Injury

In some aspects, the disclosure provides a method of distinguishing a stable kidney allograft from a kidney allograft afflicted by an alloimmune injury comprising: (a) obtaining a urine sample of a subject that received a kidney allograft; (b) detecting a panel of metabolites in the urine sample of the subject by mass spectroscopy analysis of the urine sample of the subject; and (c) distinguishing the kidney allograft afflicted by the alloimmune injury by inputting a level of the panel of metabolites detected by the mass spectroscopy analysis into a predictive model of a machine learning algorithm.

In this analysis, a 9-metabolite VSURF panel had an AUC of 95.0 in distinguishing alloimmune injury with 95% sensitivity and 76% specificity. A panel consisting of 11 metabolites for AR prediction has an AUC of 98.5 with 92.9% sensitivity and 96.3% specificity. A 5-metabolite marker panel is able to identify BKVN from non-BKVN with 92.9% sensitivity and 96.9 specificity. Through comprehensive analysis of the urine metabolome through these methods, the disclosure provides metabolite marker panels that are sensitive enough to distinguish alloimmune injury, acute rejection, and BK virus nephropathy. Because different allograft injuries can be diagnosed with great accuracy, a treatment that is effective for each condition can be prescribed. According to one aspect, the method comprises the step of characterizing a plurality of metabolites in a urine sample to obtain a metabolic profile of the sample. A panel of metabolites in the metabolic profile (i.e., a subset of the metabolic profile) can then be analyzed. The panel of metabolites can be a 3-metabolite panel, a 4-metabolite panel, a 5-metabolite panel, a 6-metabolite panel, a 7-metabolite panel, a 8-metabolite panel, a 9-metabolite panel, a 10-metabolite panel, a 11-metabolite panel, a 12-metabolite panel, a 13-metabolite panel, a 14-metabolite panel, a 15-metabolite panel, a 16-metabolite panel, a 17-metabolite panel, a 18-metabolite panel, a 19-metabolite panel, a 20-metabolite panel, a 21-metabolite panel, a 22-metabolite panel, a 23-metabolite panel, a 24-metabolite panel, a 25-metabolite panel, a 26-metabolite panel, a 27-metabolite panel, a 28-metabolite panel, a 29-metabolite panel, a 30-metabolite panel, a 31-metabolite panel, a 32-metabolite panel, a 33-metabolite panel, a 34-metabolite panel, a 35-metabolite panel, a 36-metabolite panel, a 37-metabolite panel, a 38-metabolite panel, a 39-metabolite panel, a 40-metabolite panel, a 41-metabolite panel, a 42-metabolite panel, a 43-metabolite panel, a 44-metabolite panel, a 45-metabolite panel, a 46-metabolite panel, a 47-metabolite panel, a 48-metabolite panel, a 49-metabolite panel, a 50-metabolite panel, a 52-metabolite panel, a 53-metabolite panel, a 54-metabolite panel, a 55-metabolite panel, a 56-metabolite panel, a 57-metabolite panel, a 58-metabolite panel, a 59-metabolite panel, a 60-metabolite panel, a 61-metabolite panel, a 62-metabolite panel, a 63-metabolite panel, a 64-metabolite panel, a 65-metabolite panel, a 66-metabolite panel, a 67-metabolite panel, a 68-metabolite panel, a 69-metabolite panel, a 70-metabolite panel, a 71-metabolite panel, a 72-metabolite panel, a 73-metabolite panel, a 74-metabolite panel, a 75-metabolite panel, a 76-metabolite panel, a 77-metabolite panel, a 78-metabolite panel, a 79-metabolite panel, a 80-metabolite panel, a 81-metabolite panel, a 82-metabolite panel, a 83-metabolite panel, a 84-metabolite panel, a 85-metabolite panel, a 86-metabolite panel, a 87-metabolite panel, a 88-metabolite panel, a 89-metabolite panel, a 90-metabolite panel, a 91-metabolite panel, a 92-metabolite panel, a 93-metabolite panel, a 94-metabolite panel, a 95-metabolite panel, a 96-metabolite panel, a 97-metabolite panel, a 98-metabolite panel, a 99-metabolite panel, a 100-metabolite panel, a 101-metabolite panel,

103-metabolite panel, a 104-metabolite panel, a 105-metabolite panel, a 106-metabolite panel, a 107-metabolite panel, a 108-metabolite panel, a 109-metabolite panel, a 110-metabolite panel, a 111-metabolite panel, a 112-metabolite panel, a 113-metabolite panel, a 114-metabolite panel, a 115-metabolite panel, a 116-metabolite panel, a 117-metabolite panel, a 118-metabolite panel, a 119-metabolite panel, a 120-metabolite panel, a 121-metabolite panel, a 122-metabolite panel, a 123-metabolite panel, a 124-metabolite panel, a 125-metabolite panel, a 126-metabolite panel, a 127-metabolite panel, a 128-metabolite panel, a 129-metabolite panel, a 130-metabolite panel, a 131-metabolite panel, a 132-metabolite panel, a 133-metabolite panel, a 134-metabolite panel, a 135-metabolite panel, a 136-metabolite panel, a 137-metabolite panel, a 138-metabolite panel, a 139-metabolite panel, a 140-metabolite panel, a 141-metabolite panel, a 142-metabolite panel, a 143-metabolite panel, a 144-metabolite panel, a 145-metabolite panel, a 146-metabolite panel, a 147-metabolite panel, a 148-metabolite panel, a 149-metabolite panel, a 150-metabolite panel, a 152-metabolite panel, a 153-metabolite panel, a 154-metabolite panel, a 155-metabolite panel, a 156-metabolite panel, a 157-metabolite panel, a 158-metabolite panel, a 159-metabolite panel, a 160-metabolite panel, a 161-metabolite panel, a 162-metabolite panel, a 163-metabolite panel, a 164-metabolite panel, a 165-metabolite panel, a 166-metabolite panel, a 167-metabolite panel, a 168-metabolite panel, a 169-metabolite panel, a 170-metabolite panel, a 171-metabolite panel, a 172-metabolite panel, a 173-metabolite panel, a 174-metabolite panel, a 175-metabolite panel, a 176-metabolite panel, a 177-metabolite panel, a 178-metabolite panel, a 179-metabolite panel, a 180-metabolite panel, a 181-metabolite panel, a 182-metabolite panel, a 183-metabolite panel, a 184-metabolite panel, a 185-metabolite panel, a 186-metabolite panel, a 187-metabolite panel, a 188-metabolite panel, a 189-metabolite panel, a 190-metabolite panel, a 191-metabolite panel, a 192-metabolite panel, a 193-metabolite panel, a 194-metabolite panel, a 195-metabolite panel, a 196-metabolite panel, a 197-metabolite panel, a 198-metabolite panel, a 199-metabolite panel, a 200-metabolite panel, a 201-metabolite panel, 203-metabolite panel, a 204-metabolite panel, a 205-metabolite panel, a 206-metabolite panel, a 207-metabolite panel, a 208-metabolite panel, a 209-metabolite panel, a 210-metabolite panel, a 211-metabolite panel, a 212-metabolite panel, a 213-metabolite panel, a 214-metabolite panel, a 215-metabolite panel, a 216-metabolite panel, a 217-metabolite panel, a 218-metabolite panel, a 219-metabolite panel, a 220-metabolite panel, a 221-metabolite panel, a 222-metabolite panel, a 223-metabolite panel, a 224-metabolite panel, a 225-metabolite panel, a 226-metabolite panel, a 227-metabolite panel, a 228-metabolite panel, a 229-metabolite panel, a 230-metabolite panel, a 231-metabolite panel, a 232-metabolite panel, a 233-metabolite panel, a 234-metabolite panel, a 235-metabolite panel, a 236-metabolite panel, a 237-metabolite panel, a 238-metabolite panel, a 239-metabolite panel, a 240-metabolite panel, a 241-metabolite panel, a 242-metabolite panel, a 243-metabolite panel, a 244-metabolite panel, a 245-metabolite panel, a 246-metabolite panel, a 247-metabolite panel, a 248-metabolite panel, a 249-metabolite panel, a 250-metabolite panel, a 252-metabolite panel, a 253-metabolite panel, a 254-metabolite panel, a 255-metabolite panel, a 256-metabolite panel, a 257-metabolite panel, a 258-metabolite panel, a 259-metabolite panel, a 260-metabolite panel, a 261-metabolite panel, a 262-metabolite panel, a 263-metabolite panel, a 264-metabolite panel, a 265-metabolite panel, or a 266-metabolite panel may be used in the analysis. However, as discussed in the examples, the 9-metabolite panel was nearly identical in accuracy to the full 266-metabolite model in distinguishing alloimmune injury, which has an AUC of 95.4. The 4-panel of metabolites used in the BKVN predictive model was also nearly identical to the full 266-metabolite model in distinguishing BKVN.

In one aspect of the invention, a method is provided for noninvasive detection of kidney disease, or renal complications of the of kidney allograft. The method includes measuring a change in disease specific metabolic biomarkers in a urine sample over a period-of-time. Although the disease need not be limited to kidney disease, it typically will be an alloimmune kidney disease. Living donor kidneys may require 3-5 days to reach normal functioning levels, while cadaveric donations may require 7-15 days. In some aspects, the subject provides a urine sample 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 30 days, or another suitable time period after receiving the kidney allograft.

According to another aspect, the method may further comprise monitoring by repeatedly considering, over time, the panel of metabolites present in the metabolic profile to assess stability of the allograft. In some respects, the subject provides a urine sample daily for a period of time of 1 week, 2 weeks, 3 weeks, 4 weeks, 5 weeks, 6 weeks, 7 weeks, 8 weeks, 9 weeks, 10 weeks, or another suitable period of time after receiving the transplant. In some instances, the subject provides a urine sample weekly for a period of time of 1 week, 2 weeks, 3 weeks, 4 weeks, 5 weeks, 6 weeks, 7 weeks, 8 weeks, 9 weeks, 10 weeks, 11 weeks, 12 weeks, 3 or another suitable period of time after receiving the transplant. In other instances, the subject provides a urine sample monthly during the life of the subject for the monitoring of the stability of the kidney (lifelong monitoring). Lifelong monitoring may provide an insight on the stability of the allograft, particularly when the subject experiences a change in treatment regimen (e.g., when a calcineurin inhibitor, an immunosuppressant, or a corticosteroid is added/removed from the treatment regimen).

According to another aspect, the method may further comprise statistically analyzing differences between the metabolic profile and reference profile to identify at least one biomarker. Biomarkers or a group of biomarkers having a significance level of less than 80%, 85%, 90%, 95%, may be rejected from the predictive model. Biomarkers having a significance level greater than 70%, 75%, 80%, 85%, 90%, 91%, 92%, 93%, 94%, 05%, 06%, 07%, 98%, or 99% may be included in the predictive model.

According to an aspect, the disclosure provides a method for assessing subject health comprising: providing a bodily fluid or tissue sample from a subject; collecting a metabolic profile from the bodily fluid or tissue sample, the metabolic profile comprising two or more metabolites; and comparing the metabolic profile to at least one reference profile to assess the health of the subject. The at least one reference profile profiling at least one of: one or more disease, injury or disorder. The reference profile may be established from the metabolic profile collected from subjects with biopsy matched (i.e., “known”) allograft injuries, from a healthy population, from a stable allograft injury, or both. The reference profile may be used to train a machine learning algorithm. For instance, any machine learning algorithm can be used to identify statistically significant metabolites in a urine sample. In one example, the predictive model used was Random Forests via the random Forest package in R. Significant metabolites were selected from the Random Forests model using the VSURF package in R. This is a prediction model does not have a threshold quantity or signal for a particular metabolite. However, using the known samples to train a training set on the machine learning algorithm it is possible to generate a predictive model that distinguishes various types of transplant injuries.

By the use of the novel panels of biomarkers disclosed herein, the occurrence or risk of various kidney allograft injury conditions may be assessed. A first condition that may be assessed by the methods of the invention is acute rejection (AR). Acute rejection encompasses any number of ongoing immune-mediated processes wherein the host immune system attacks the grafted tissue, with resulting effects such as cell death, necrosis, impairment of graft function, and other pathologies associated with acute rejection. AR may be distinguished from other allograft injuries by a panel of biomarkers, also referred to herein as “AR Biomarkers”. The AR biomarkers may be detected by a panel of 3- to 11-biomarkers comprising: glycine, glutaric acid, adipic acid, inulobiose, threose, sulfuric acid, taurine, N-methylalanine, asparagine, 5-aminovaleric acid lactame, and myoinositol. In various embodiments, the selected panel of AR biomarkers comprises one, two, three, four, five, six, seven, eight, nine, ten, eleven or all of the AR Biomarkers. The sensitivity and accuracy of the method may be adjusted based on the number of biomarkers selected.

An allograft injury type comprising CAN and/or AR allograft injuries may be detected by a suite of biomarkers, referred to herein as “Allograft Biomarkers.” The allograft injury biomarkers may be a 9-biomarker panel comprising: Glycine, N-methylalanine, adipic acid, glutaric acid, inulobiose, threitol, isothreitol, sorbitol, and isothreonic acid. In various embodiments, the selected panel comprises one, two, three, four, five, six, seven, eight, or all of the allograft injury biomarkers.

An allograft injury comprising BKVN infection may be distinguished from acute rejection, a stable allograft, or another general allograft injury by a panel of 4-, 5-, or more biomarkers, referred to herein as “BKVN Biomarkers.” comprising: arabinose, 2-hydroxy-2-methylbutanoic acid, hypoxanthine, benzylalcohol, and N-acetyl-D-mannosamine. Additionally, a BKVN infection may be distinguished from a stable allograft (STA) based in a panel comprising at least 4 metalbolites, for example a panel of 4-metabolites, wherein the panel includes arabinose, 2-hydroxy-2-methylbutanoic acid, octadecanol, and phosphate. In various embodiments, the selected panel comprises one, two, three, four, five or all of the BKVN Biomarkers.

It will be appreciated that in certain embodiments, other types of allograft injury, or subtypes of the selected allograft injuries are detectable using the methods of the invention. For instance, a set of biopsy matched samples afflicted with a known transplant injury may be used to train a new set of machine learning algorithms. These other types of diseases include, for example, but is not limited to, hyper-acute rejection, early acute rejection, late acute rejection, polycystic kidney disease, chronic glomerulonephritis, or Lupus nephritis.

In certain embodiments, myo-inositol is included in the selected biomarker panels. As demonstrated herein, myo-inositol is an indicator of AR and can also be used to discriminate AR, CAN/AR, and BKVN injuries from each other. When included in, for example, the BKVN panel, myo-inositol can provide additional sensitivity in distinguishing AR from BKVN.

A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more as compared to the level of the biomarker in a subject that has a stable kidney allograft. Alternatively, the level of the biomarker is present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100%, or less as compared to the level of the biomarker in a subject that has a stable kidney allograft.

In one embodiment, the sample is analyzed for a selected panel of biomarkers comprising AR biomarkers, for the detection of AR. In one embodiment, the sample is analyzed for a selected panel of biomarkers comprising CAN/AR biomarkers, for the detection of a composite of allograft injuries. In one embodiment, the sample is analyzed for a selected panel of biomarkers comprising BKVN biomarkers, for the detection of BKVN. In other embodiments, the sample is analyzed for a panel of biomarkers comprising a combination of CAN/AR and AR biomarkers for the detection of CAN/AR and AR, a combination of CAN/AR and BKVN biomarkers for the detection of CAN/AR and BKVN, a combination of AR and BKVN biomarkers, for the detection of AR and BKVN or a combination of AR, CAN/AR, and BKVN biomarkers, for the detection of AR, CAN/AR, and BKVN.

### Detection of Biomarkers in Samples

Sample Analysis. Samples, such as urine samples, are analyzed for selected panels of biomarkers described herein. Analysis encompasses measuring the presence and/or abundance of a selected panel of biomarkers, by the sample. Generally, the selected biomarkers are measured directly, however, in alternative implementations, the presence and/or abundance of the selected biomarkers is determined by the measurement of proxy species which are indicative of the selected biomarkers. The sample analysis may be carried out by any suitable method or combination of methods for measurement of the selected biomarkers. The presence and/or abundance of a biomarker from a select group of biomarkers can be determined using a number of methods, including, but not limited to ELISA, mass spectrometry (MS) methods (e.g. gas chromatography/MS, liquid chromatography/MS, inductively coupled plasma/MS, MALDI/TOF, ion mobility spectrometry/MS, SELDI/TOF, accelerator MS, tandem MS), enzymatic assays, fluorescent assays, colorimetric assays, chemiluminescent labels, chromatography, lateral flow assays, and other methods known in the art. Mass spectrometry (MS) is an analytical technique that measures the “mass” of analyte molecules in a sample by ionizing them and sorting the resultant ions based on their mass-to-charge (m/z) ratio. Combined with an upfront liquid- or gas-phase sample separation system, mass spectrometry provides one of the most effective means available for analyzing complex samples comprising a plurality of low abundance analytes, as is common, for example, in biological samples. In some embodiments, the output of the mass spec analysis is inputted into a predicted model of a machine learning algorithm.

Generally, mass spectrometers share the requirement that the ions be in the gas phase prior to introduction into a mass analyzer. A variety of sample ionization modes have been developed including, but not limited to, matrix-assisted laser desorption and ionization (MALDI) and electrospray ionization (ESI). In the MALDI technique, the sample (e.g., a urine sample comprising a mixture of biomarkers) is mixed with an energy absorbing matrix (EAM) such as sinapinic acid or -cyano-4-hydroxycinnamic acid and crystallized onto a metal plate. Surface enhanced laser desorption and ionization (SELDI) is a common variant of the technique that incorporates additional surface chemistry on the metal plate to promote specific binding of certain classes of proteins. The plate is inserted into a vacuum chamber, and the matrix crystals are struck with light pulses from a nitrogen laser. The energy absorbed by the matrix molecules is transferred to the proteins, causing them to desorb, ionize, and produce a plume of ions in the gas phase that are accelerated in the presence of an electric field and drawn into a flight tube where they drift until they strike a detector that records the time of flight. The time of flight may in turn be used to calculate the m/z ratio for the ionized species. In some embodiments of the disclosed devices, an outlet port of the device may comprise a capillary or other feature used to deposit separated analyte bands (or fractions thereof) onto a MALDI plate in preparation for mass spectrometric analysis, e.g., to correlate isoelectric points for specific analyte bands with MALDI mass spectrometer data.

Electrospray ionization (also referred to herein simply as “electrospray”) is another widely used technique due to its inherent compatibility for interfacing liquid chromatographic or electrokinetic chromatographic separation techniques with a mass spectrometer. As noted above, in electrospray ionization, small droplets of sample and solution are emitted from at a distal end of a capillary or microfluidic device comprising an electrospray feature (e.g., an emitter tip or orifice) by the application of an electric field between the tip or orifice and the mass spec source plate. The droplet then stretches and expands in this induced electric field to form a cone shaped emission (i.e., a “Taylor cone”), which comprises increasingly small droplets that evaporate and produce the gas phase ions that are introduced into the mass spectrometer for further separation and detection. Typically, emitter tips are formed from a capillary, which provides a convenient droplet volume for ESI.

In some embodiments of the disclosed methods, other ionization methods are used, such as inductive coupled laser ionization, fast atom bombardment, soft laser desorption, atmospheric pressure chemical ionization, secondary ion mass spectrometry, spark ionization, thermal ionization, and the like. With respect to electrospray ionization, in some embodiments the disclosed microfluidic devices comprise features designed to promote efficient electrospray ionization and convenient interfacing with downstream mass spectrometric analysis. The mass-to-charge ratio (or “mass”) for analytes expelled from the microfluidic device (e.g., the metabolite) and introduced into a mass spectrometer can be measured using any of a variety of different mass spectrometer designs. Examples include, but are not limited to, time-of-flight mass spectrometry, quadrupole mass spectrometry, ion trap or orbitrap mass spectrometry, distance-of-flight mass spectrometry, Fourier transform ion cyclotron resonance, resonance mass measurement, and nanomechanical mass spectrometry.

Methods of Treatment of Acute Rejection, Chronic Allograft Nephropathy, and BK virus Nephropathy in Subjects

The methods, compositions, and kits of this disclosure may comprise a method to treat, arrest, reverse, or ameliorate an allograft injury and associated conditions in a subject. The subject (also referred to as “patient”) is a kidney allograft recipient, having received at least one transplanted organ, such as a kidney transplant. The subject may comprise any subject at risk of allograft kidney injury, including post-operative subjects, subjects displaying symptoms of potential allograft injury, or subjects otherwise at risk of allograft injury. The subject may be a human subject, or non-human animal, such as a test animal or veterinary subject. In one embodiment, the subject is a pediatric subject, for example a subject of 18 years of age or under. In another embodiment, the subject is a youth subject, for example a subject of 18-25 years of age. In some instances, the subject may have received more than one organ transplant.

In some cases, the disease may be an Acute Rejection, Chronic Allograft Nephropathy, or BK Virus Nephropathy. In some cases, the treatment is achieved by administrating a therapeutically-effective dose of an immunosuppressant drug when, or after, the alloimmune injury is detected. In some cases, the immunosuppressive drug is a calcineurin inhibitor, such as cyclosporin. In other cases, the immunosuppressive drug is belatacept. In some instances, the immunosuppressive drug is a lymphocyte depleting antibody, such as Thymoglobulin. In other instances, the immunosuppressive drug is mycophenolate or azathioprine. In some instances, the drug is a corticosteroid. Yet in other instances, the method further comprises administering an effective amount of an intravenous immunoglobulin when the alloimmune injury is detected in the subject.

Treatment may be provided to the subject before clinical onset of disease. For example, treatment can be provided to the subject after the detection of the biomarker panel, but before the subject manifest's symptoms of allograft injury. Treatment may be provided to the subject after clinical onset of allograft injury. Treatment may be provided to the subject after 1 day, 1 week, 6 months, 12 months, or 2 years after clinical onset of the allograft injury. Treatment may be provided to the subject for more than 1 day, 1 week, 1 month, 6 months, 12 months, 2 years or more after clinical onset of allograft injury. Treatment may be provided to the subject for less than 1 day, 1 week, 1 month, 6 months, 12 months, or 2 years after clinical onset of the allograft injury. Treatment may also include treating a human in a clinical trial. A treatment can comprise administering to a subject a pharmaceutical composition, such as one or more of the pharmaceutical compositions described throughout the disclosure. A subject may be monitored for allograft injury, and a treatment may be administered, at any point during the life of the subject.

The methods of the invention may be applied by the use of various biological samples. In a primary embodiment, the sample is a urine sample. Urine samples may advantageously be collected in a non-invasive manner, and in a non-clinical setting. Being the direct form of kidney output, urine samples are uniquely able to capture renal biomarkers. Exemplary urine samples include self-collected samples or samples collected in a clinical setting. Exemplary urine samples include first or second void daily samples, for example, mid-stream samples, collected as known in the art. Urine samples may be processed by techniques suitable for the analytical methods to be applied. For example, urine samples may be centrifuged to remove particulate and cellular material. Samples may be frozen or lyophilized.

It will be understood that the methods of the invention may be applied to other sample types, including any biological or waste material derived from the subject, including, blood, plasma, saliva, biopsy material, tissue, and cell preparations from the subject, as the biomarkers may be present in other sample types.

In one example, urine samples (e.g. void mid-stream urine) may be collected from a subject. Supernatant of the urine may be collected after centrifugation, and lyophilized. Further steps include, for example, addition of an internal standard, and addition of a derivatization agent. Steps may further include analyzing the sample using a gas chromatography/MS system. In certain embodiments, the presence and/or abundance of in the biological sample are compared with an internal standard added during the processing steps. In certain embodiments, the presence and/or abundance of certain compounds compared with a biological standard, for instance, the presence of a compound shared among other biological samples in the predictive model. An example of a biological standard for a urine sample may include, for example, creatinine, although it will be appreciated that other standards may be used.

The suitable treatment will be selected based on the standard of care for the selected injury. For example, in the treatment of AR and composite types of rejection (e.g., CAN/AR), appropriate immunosuppressive therapies may be given to a subject, including augmentation of maintenance immunosuppression. Examples of the immunosuppressive therapies include, for example, treatment with corticosteroids, calcineurin inhibitors, anti-proliferatives, mTOR inhibitors, monoclonal anti-IL-2Ra Receptor antibodies, polyclonal anti-T-cell antibodies, monoclonal anti-CD20 antibodies, and immunosuppressive fusion proteins (e.g. belatacept). In the case of BKVN, reduction of immunosuppression may be utilized to treat the viral infection, and/or the administration of antiviral drugs and other BKVN treatments known in the art.

In some embodiments, an intermediate step is performed to confirm or differentiate the allograft injury. For example, in one embodiment, a biopsy or other diagnostic test is performed on the subject to confirm that the detected injury is occurring. In one embodiment, wherein the selected injury is CAN/AR, additional diagnostic methods may be applied to differentiate between CAN and AR such that appropriate treatment can be selected.

### Predictive Model Analysis of Metabolite Data

Metabolite data (data representing the kidney disease status) can be used to classify a sample. The challenge in using metabolite data for the generation of a predictive model is to remove irrelevant variables, to select all important ones, and to determine a sufficient subset for prediction. Here, mass spec analysis was conducted in a sufficiently large sample set of human urine with matched biopsies. The mass spec analysis identified over 300 metabolites as being present in each matched biopsy urine sample, which allowed for precise and unambiguous definition of a training set in a nonparametric statistical method. By the measurements obtained of the selected panel of biomarkers, the occurrence, absence, and/or likelihood of the subject having a selected allograft kidney injury can be determined. In one case, a predictive model is applied to biomarker measurements in order to determine the occurrence, absence, and/or likelihood of the subject having the selected kidney allograft injury. In some instances, the severity of the selected allograft injury is assessed by the biomarkers.

The predictive models of the invention are used to assess the rejection status or the risk of rejection. The predictive models of the invention may be constructed using post-hoc analysis of biomarker data in subjects having known rejection outcomes, for example as described in the Examples and descriptions provided herein. The predictive model may be any mathematical model, which determines a type of rejection using a biomarker profile (independent variables). The predictive model may be generated using statistical methods such as: logistic regression analysis, linear discriminate analysis, partial least squares-discriminate analysis, multiple linear regression analysis, multivariate non-linear regression, backwards stepwise regression, generalized additive models, supervised and unsupervised learning models, cluster analysis, and other statistical model generating methods known in the art. Subsets of historical data may be utilized to generate, train, or validate the model, as known in the art.

In one embodiment, the model is based upon the number of indicative biomarkers present. A biomarker may be deemed “present” if it is measured at a detectable level, or measured at an abundance that exceeds a selected threshold level (e.g. a normal or stable threshold value). “Elevated” may also be determined in comparison with the abundance of the biomarker in the same subject from an earlier time point (e.g. a time point at which the subject was considered stable). In other implementations, the measured biomarker values are assigned weighted values reflective of their relative contributions to rejection. Desired levels of specificity and sensitivity are selected in constructing the model. The model may also account for other variables relevant to disease status, such as donor and recipient age, sex, race, relative date before or after transplant surgery that that the sample was measured, the source of the allograft (e.g. organ from a living relative, organ from a living non-relative), and number of previous transplants. Separate models may be generated for each selected allograft injury type, e.g. AR/CAN, AR, and BKVN, or integrated models may be used when relevant biomarkers from two or more injury types are measured.

The output of the model will be a score, for example, a qualitative score or a quantitative score. In one embodiment, the output of the predictive model is an index score, being a value that can be compared to a defined range reflective of allograft injury status. In one embodiment, the output of the model is a probability score, for example, a probability of the selected allograft injury types occurring, for example, a probability of AR, AR/CAN, or BKVN. In one embodiment, the output of the model is a classification, for example classification of the subject being at low-risk, intermediate risk, or high risk for AR, AR/CAN, or BKVN. In one embodiment, the output of the model is a classification, for example classification of the subject being at negative or positive for AR, AR/CAN, or BKVN, for example, at selected sensitivity and specificity cutoff values. In one embodiment, the output of the model is a score, for example a score reflecting the severity of the selected allograft injury, if present.

Many statistical classification techniques are known to those of skill in the art. In supervised learning approaches, a group of samples from two or more groups (e.g. contaminated with a pathogen and not) are analyzed with a statistical classification method. Allograft injury presence/absence data—or data that distinguishes one type of allograft injury from another—can be used as a classifier that differentiates between the two or more groups. A new sample can then be analyzed so that the classifier can associate the new sample with one of the two or more groups. Commonly used supervised classifiers include without limitation the neural network (multi-layer perceptron), support vector machines, k-nearest neighbours, Gaussian mixture model, Gaussian, naive Bayes, decision tree and radial basis function (RBF) classifiers. Linear classification methods include Fisher's linear discriminant, logistic regression, naive Bayes classifier, perceptron, and support vector machines (SVMs). Other classifiers for use with the invention include quadratic classifiers, k-nearest neighbor, boosting, decision trees, random forests, neural networks, pattern recognition, Bayesian networks and Hidden Markov models. One of skill will appreciate that these or other classifiers, including improvements of any of these, are contemplated within the scope of the invention.

In several instances, a random forest or random decision forests were executed on a computer to analyze the metabolite data retrieved from the urine samples. Random forests (abbreviated RF in the sequel) are an attractive nonparametric statistical method to deal with these problems, because RF models are based on decision trees and use aggregation ideas, which allow one to consider different predictive models and problems, namely regression, two-class and multiclass classifications. Random forests or random decision forests are an ensemble learning method for classification, regression and other tasks that operates by constructing a multitude of decision trees at training time and outputting the class that is the mode of the classes (classification) or mean prediction (regression) of the individual trees. Notably, random decision forests correct for decision trees' habit of overfitting to their training set.

Classification using supervised methods is generally performed by the following methodology:

In order to solve a given problem of supervised learning (e.g. learning to recognize allograft injury) one has to consider various steps:
1. Gather a training set. These can include, for example, samples that are from subjects that afflicted with AR, samples from subjects afflicted with CAN, samples from subjects afflicted with BK virus injury, samples that are from subjects that have a stable allograft, and optionally samples from subjects that have never received an allograft. The training samples are used to “train” the classifier.
2. Determine the input “feature” representation of the learned function. The accuracy of the learned function depends on how the input object is represented. Typically, the input object is transformed into a feature vector, which contains a number of features that are descriptive of the object. The number of features should not be too large, because of the curse of dimensionality; but should be large enough to accurately predict the output. The features might include a set of metabolites present or missing in a subjected afflicted with AR as compared to a subject that has received a stable transplant as described herein.
3. Determine the structure of the learned function and corresponding learning algorithm. A learning algorithm is chosen, e.g., artificial neural networks, decision trees, Bayes classifiers or support vector machines. The learning algorithm is used to build the classifier.
4. Build the classifier (e.g. classification model). The learning algorithm is run on the gathered training set. Parameters of the learning algorithm may be adjusted by optimizing performance on a subset (called a validation set) of the training set, or via cross-validation. After parameter adjustment and learning, the performance of the algorithm may be measured on a test set of naive samples that is separate from the training set.

Once the classifier (e.g. classification model) is determined as described above, it can be used to classify a sample, e.g., that of a subject that is being monitored for an allograft rejection by providing a urine sample for metabolite analysis.

Unsupervised learning approaches can also be used with the invention. Clustering is an unsupervised learning approach wherein a clustering algorithm correlates a series of samples without the use the labels. The most similar samples are sorted into “clusters.” A new sample could be sorted into a cluster and thereby classified with other members that it most closely associates.

In many aspects, the systems, platforms, software, networks, and methods used for the analysis of the mass spec data and predictive model include one or more non-transitory computer readable storage media encoded with a program including instructions executable by the operating system of an optionally networked digital processing device. For instance, in some aspects, the methods comprise creating data files associated with a plurality of metabolites from a plurality of samples associated with subject that received an allograft. In further embodiments, a computer readable storage medium is a tangible component of a digital processing device.

In some embodiments, the systems, platforms, software, networks, and methods disclosed herein include at least one computer program that is used in the analysis of the mass spec data and/or prediction model. A computer program includes a sequence of instructions, executable in the digital processing device's CPU, written to perform a specified task. In light of the disclosure provided herein, those of skill in the art will recognize that a computer program may be written in various versions of various languages. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

The systems, platforms, software, networks, and methods disclosed herein include, in various embodiments, software, server, and database modules that are used in the analysis of mass spec data. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

As described herein, targeted metabolomic analyses of biopsy-matched urine samples enabled the generation of refined metabolite panels that can non-invasively detect graft injury phenotypes with high confidence. These urine biomarkers can be rapidly assessed for early diagnosis of specific transplant injuries, opening the window for predictive, precision transplant medicine.

A plurality of predictive models may be created, in some instances generating distinct panels of metabolites for the distinction of various types of allograft injuries. High-dimensional data, such as metabolite detection data, can be difficult to interpret. One approach to simplification is to assume that the data of interest lie on an embedded non-linear manifold within the higher-dimensional space. If the manifold is of low enough dimension, the data can be visualized in the low-dimensional space. Principal curves and manifolds give the natural geometric framework for nonlinear dimensionality reduction and extend the geometric interpretation of PCA by explicitly constructing an embedded manifold, and by encoding using standard geometric projection onto the manifold. Non-limiting examples of manifold learning algorithms that may be used to create predictive models for metabolite data analysis include SDD Maps, isomap, locally-linear embedding, laplacian eigenmaps, Sammon's mapping, self-organizing map, principal curves and manifolds, autoencoders, Gaussian process latent variable models, contagion maps, curvilinear component analysis, curvilinear distance analysis, diffeomorphic dimensionality reduction, Kernel principal component analysis, manifold alignment, diffusion maps, Hessian Locally-Linear Embedding (Hessian LLE), Modified Locally-Linear Embedding (MLLE), relational perspective map, local tangent space alignment, local multidimensional scaling, Maximum variance unfolding, nonlinear PCA, Data-driven high-dimensional scaling, manifold sculpting, t-distributed stochastic neighbor embedding, RankVisu, topologically constrained isometric embedding, uniform manifold approximation and projection. Related linear decomposition methods include independent component analysis (ICA), principal component analysis (PCA), singular value decomposition (SVD), or factor analysis.

### Kits for the Detection of Metabolite Panels

Provided herein are novel biomarkers as indicators for kidney allograft injury. Accordingly, these discoveries enable the design of integrated assays to simultaneously measure disease indicators in a streamlined process. Provided herein are assay kits based on the panel of biomarkers identified in the disclosure that can be used to facilitate the fast, inexpensive, and convenient assessment of allograft injury biomarker profiles in a sample. As used herein, an “assay kit” will refer to an aggregated collection of products that can be used to quantify two or more allograft rejection biomarkers of the invention in a sample.

The assay kit will comprise a plurality of detection/quantification tools specific to each biomarker detected by the kit. Many of the biomarkers disclosed herein comprise metabolites which may be detected by immunoassays or like technologies. The detection/quantification tools may comprise capture ligands of multiple types, each directed to the selective capture of a specific biomarker in the sample. The detection/quantification tools may comprise labeling ligands of multiple types, each directed to the selective labeling of a specific biomarker in the sample, for example, comprising enzymatic, fluorescent, or chemiluminescent labels for the quantification of target species. For example, the capture and/or labeling ligands may comprise antibodies, affibodies, aptamers, riboswitches or other moieties that specifically bind to a selected biomarker. The assay kit may further comprise labeled secondary antibodies, for example comprising enzymatic, fluorescent, or chemiluminescent labels and associated reagents.

In one embodiment, the assay kit comprises the physical elements of a quantitative multiplex assay, for example a direct assay, an indirect assay, a sandwich assay, or a competitive assay, as known in the art, for example, an ELISA assay, wherein the assay elements enable the detection of multiple allograft rejection biomarkers.

In one embodiment, the assay kits of the invention comprise reagents or enzymes, which create quantifiable signals based on concentration dependent reactions with biomarker species in the sample. For example, sugar panel analysis may employ procedures to detect certain carbohydrates (e.g. L-arabinose/D-galactose assays for detecting arabinose levels).

Assay kits may further comprise elements such as reference standards of the biomarkers to be measured, washing solutions, buffering solutions, reagents, printed instructions for use, and containers). The assay kit may include urine collection cups and sample processing tools and reagents. The diagnostic tools of the invention may comprise lab-on-chip or microfluidic devices for sample analysis. The assay kits may further encompass software, e.g. non-transitory computer readable storage medium comprising a set of instructions which carry out the application of the predictive model to analyze measured biomarker values.

In one embodiment, the assay kit of the invention is directed to the quantification of two or more renal transplant disease biomarkers. In one embodiment, the assay kit of the invention is directed to the quantification of two or more CAN/AR biomarkers to detect AR/CAN: N-methylalanine, adipic acid, glutaric acid, inulobiose, threitol, isothreitol, sorbitol, and isothreonic acid. In one embodiment, the assay kit of the invention is directed to the quantification of two or more AR biomarkers to detect AR: glycine, glutaric acid, adipic acid, inulobiose, threose, sulfuric acid, taurine, N-methylalanine, asparagine, and 5-aminovaleric acid lactame. In one embodiment, the assay kit of the invention is directed to the quantification of two or more BKVN biomarkers to detect BKVN: arabinose, 2-hydroxy-2-methylbutanoic acid, hypoxanthine; benzylalcohol, and N-acetyl-D-mannosamine.

In some instances, provided herein is a method of distinguishing a stable kidney allograft from an injured kidney allograft in a subject that received a kidney allograft comprising: a) contacting i) a urine sample from said subject, and ii) reagents for detection of metabolites associated with a pre-determined kidney allograft injury; b) reacting said reagents with said urine sample; and c) determining an amount of glycine, N-methylalanine, and inulobiose in said urine sample using said reagents; wherein if the amount of the metabolites is above a cut-off level, the said subject is determined to have an injured kidney allograft.

### EXAMPLES

### Example 1. Comprehensive Urine Metabolomics Identifies Metabolites Specific to Distinct Renal Allograft Injuries

Despite new advancements in surgical tools and therapies, exposure to immunosuppressive drugs related to non-immune and immune injuries can cause slow deterioration and premature failure of organ transplants. Prediction of these injuries by non-invasive urine monitoring would be a significant clinical advancement for patient management. The Examples disclose herein describe the metabolomic profiles of biopsy matched urine samples from 310 unique kidney transplant recipients using gas chromatography-mass spectrometry (GC-MS). Using this approach, focused metabolite panels were identified that could detect biopsy confirmed acute rejection with 92.9% sensitivity and 96.3% specificity (11 metabolites) and could differentiate BK viral nephritis (BKVN) from acute rejection with 88.9% sensitivity and 94.8 specificity (4 metabolites).

### Subjects and Samples:

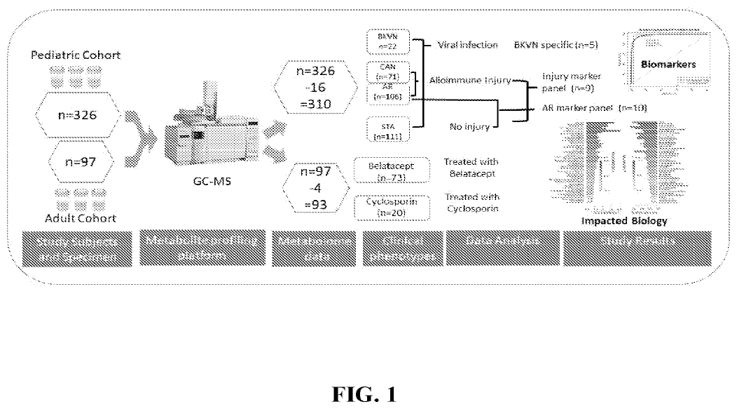

Biobanked urine samples from the Department of Surgery of the University of California San Francisco were screened for their matching with biopsy data on the day of urine collection. Out of a total 2016 banked urine samples 770 were biopsy matched. 326 unique biopsy matched, and clinically annotated untreated urine samples were included in the first part of this study. 16 pediatric samples had missing data on more than one third of total metabolites identified and were excluded from further analyses (see FIG. 1). Baseline characteristic of the study subjects is provided in Table 1.

TABLE 1
Patient demographic data for the pediatric cohort

| Phenotype | AR | STA | CAN | BKVN |
|---|---|---|---|---|
| Number of Patients | 106 | 111 | 71 | 22 |
| Maintenance (% Steroid- | 63.2% | 50.5% | 56.3% | 36.4% |
| free) | | | | |
| Recipient Gender (% M) | 64.2% | 58.6% | 67.6% | 59.1% |
| Recipient Age* (years) | 13 ± 5 | 14 ± 5 | 10 ± 6 | 14 ± 5 |
| | (14; 2-21) | (15; 1-21) | (10; 1-20) | (17; 1-18) |
| Donor Gender (% M) | 46.2% | 52.3% | 52.1% | 72.7% |
| Donor Age* (years) | 29 ± 11 | 30 ± 10 | 30 ± 10 | 28 ± 10 |
| | (29; 4-50) | (28; 14-51) | (32; 12-50) | (29; 16-49) |
| Month post-Txp | 71 ± 32 | 15 ± 24 | 23 ± 32 | 8 ± 7 |
| (mean ± SD) | | | | |
| Donor Source (%): | | | | |
| 1 = Living Related | 1 = 24.5% | 1 = 37.8% | 1 = 43.7% | 1 = 9.1% |
| 2 = Living Unrelated | 2 = 40.6% | 2 = 8.1% | 2 = 8.5% | 2 = 31.8% |
| 3 = Cadaveric | 3 = 34.0% | 3 = 44.1% | 3 = 47.9% | 3 = 54.5% |
| Recipient Race (%) | | | | |
| 1 = Caucasian | 1 = 42.5% | 1 = 43.2% | 1 = 50.7% | 1 = 27.3% |
| 2 = Asian | 2 = 5.7% | 2 = 4.5% | 2 = 7.0% | 2 = 0.0% |
| 3 = African American | 3 = 16.0% | 3 = 18.0% | 3 = 18.3% | 3 = 13.6% |
| 4 = Hispanic | 4 = 7.5% | 4 = 2.7% | | |
| 5 = Mixed and Others | 5 = 12.3% | 5 = 16.2% | 5 = 9.9% | 5 = 0.0% |

AR, acute rejection;
STA, stable graft function;
CAN, chronic allograft nephropathy;
BKVN, BK virus nephropathy,
*Age in years: mean + SD(median; range)

In addition, 97 adult urine samples were collected as a part of a Bristol Myers Squibb (BMS) clinical trial for belatacept were included. Table 2 describes the demographic data for the treated cohort.

TABLE 2
Patient demographic for the adult cohort

| Treatment | Belatacept | Cyclosporin |
|---|---|---|
| Number of Patients | 73 | 20 |
| Recipient Gender (% M) | 66.4% | 73.1% |
| Recipient Age* (years) | 51 ± 13 | 49 ± 16 |
| | (52; 18-80) | (49; 25-71) |
| Donor Gender (% M) | 48.7% | 69.2% |
| Donor Age* (years) | 51 ± 14 | 45 ± 18 |
| | (55; 16-75) | (47; 12-81) |
| Months Post-Txp (mean ± SD) | 27 ± 1 | 24 ± 8 |
| Donor Source (%): | | |
| 1 = Living | 1 = 15.9% | 1 = 11.5% |
| 2 = Cadaveric | 2 = 84.1% | 2 = 88.5% |

*Age in years: mean + SD (median; range)

The biopsies were read by a central pathologist and scored by the Banff and Chronic Allograft Damage Index (CADI) as acute cellular or humoral rejection with clinical graft dysfunction and tubulitis and/or vasculitis on histology (AR; n=106), stable with no histological or clinical graft injury (STA; n=111), chronic allograft nephropathy with clinical graft dysfunction and chronic tubule-interstitial injury on histology (CAN; n=69), and BK viral nephritis with SV40 staining on histology, with/without clinical graft dysfunction (BKVN; n=22). “Graft injury” in this study was defined as a greater than 20% increase in serum creatinine from its previous steady-state baseline value and an associated biopsy that was pathological. “Acute rejection” (AR) was defined at minimum, as per Banff Schema, a tubulitis score>1 accompanied with an interstitial inflammation score>1. “Chronic allograft nephropathy” (CAN) was defined at minimum as a tubular atrophy score>1 accompanied by an interstitial fibrosis score>1. “BK virus nephropathy” (BKVN) was defined as positivity of polyomavirus PCR in peripheral blood, together with a positive SV40 stain in the concomitant renal allograft biopsy. “Stable kidney grafts with normal protocol biopsies” (STA) allografts were defined by an absence of significant injury pathology as defined by Banff schema.

All samples were collected from pediatric and young adult recipients transplanted between 2000-2011 at Lucile Packard Children's Hospital of Stanford University, under IRB approved protocols. The study was also approved by The Human Research Protection Program (HRPP) of the University of California, San Francisco to allow analysis of biobanked samples. All patients/guardians provided informed consent to participate in the research, in full adherence to the Declaration of Helsinki. The clinical and research activities being reported are consistent with the Principles of the Declaration of Istanbul as outlined in the ‘Declaration of Istanbul on Organ Trafficking and Transplant Tourism.

The overall study design is summarized in FIG. 1.

### Example 2. Sample Collection and Gas Chromatography-Mass Spectrometry (GS-MS) Analysis

Urine collection, initial processing, storage, and GS-MS analysis: Second morning void mid-stream urine (50-100 mL) was collected in sterile containers and was centrifuged at 2000×g for 20 min at room temperature within 1 hr of collection. The supernatant was separated from the pellet containing any particulate matter including cells and cell debris. The pH of the supernatant was adjusted to 7.0 and stored at −80° C. until further analysis. The derivatization procedure has been performed with standard-of-care procedures. Briefly, neat urine samples were lyophilized without further pretreatment after our initial finding of severe alterations using urease treatments. To the dried samples, 2011.1 of 40 mg/ml methoxylamine hydrochloride in pyridine was added, and samples were agitated at 30° C. for 30 min. Subsequently, 18011.1 of trimethylsilylating agent N-methyl-N-trimethylsilyltrifluoroacetamide (MSTFA) was added, and samples were agitated at 37° C. for 30 min.

Gas chromatography-mass spectrometry (GC-MS) is an analytical method that combines the features of gas-chromatography and mass spectrometry to identify different substances within a test sample. GC-MS analysis was performed using a Agilent 6890 N gas chromatograph (Atlanta, Ga., USA) interfaced to a time-of-flight (TOF) Pegasus III mass spectrometer (Leco, St. Joseph, Mich., USA). Automated injections were performed with a programmable robotic Gerstel MPS2 multipurpose sampler (Millheim an der Ruhr, Germany). The GC was fitted with both an Agilent injector and a Gerstel temperature-programmed injector, cooled injection system (model CIS 4), with a Peltier cooling source. An automated liner exchange (ALEX) designed by Gerstel was used to eliminate cross-contamination from sample matrix occurring between sample runs. Multiple baffled liners for the GC inlet were deactivated with 111.1 injections of MSTFA. The Agilent injector temperature was held constant at 250° C. while the Gerstel injector was programmed (initial temperature 50° C., hold 0.1 min, and increased at a rate of 10° C./s to a final temperature of 330° C., hold time 10 min). Injections of 111.1 were made in split (1:5) mode (purge time 120 s, purge flow 40 ml/min). Chromatography was performed on anRtx-5Si1 MS column (30 m×0.25 mm i.d., 0.25 μm film thickness) with an Integra-Guard column (Restek, Bellefonte, Pa., USA). Helium carrier gas was used at a constant flow of 1 ml/min. The GC oven temperature program was 50° C. initial temperature with 1 min hold time and ramping at 20° C./min to a final temperature of 330° C. with 5 min hold time. Both the transfer line and source temperatures were 250° C. After a solvent delay of 350 s, mass spectra were acquired at 20 scans/s with a mass range of 50 to 500 m/z. Initial peak detection and mass spectrum deconvolution were performed with Chroma-TOF software (version 2.25, Leco), and later samples were exported to the netCDF format for further data evaluation with MZmine and XCMS.

The detailed information on the metabolites and structure in terms of binbase ID, KEGG ID, and PubChem ID is provided in Table 3.

TABLE 3
266 Metabolites Identified in Urine

| | | ret | quant | BinBase | KEGG | PubChem |
|---|---|---|---|---|---|---|
| | BinBase name | index | mz | id | id | id |
| 1 | hippuric acid | 616499 | 105 | 275367 | C01586 | 464 |
| 2 | glycine | 364262 | 174 | 227957 | C00037 | 750 |
| 3 | mannitol | 665209 | 103 | 210512 | C00392 | 6251 |
| 4 | galactitol | 668930 | 319 | 200575 | C01697 | 453 |
| 5 | taurine | 557250 | 326 | 234595 | C00245 | 1123 |
| 6 | citric acid | 617723 | 273 | 217681 | C00158 | 311 |
| 7 | oxoproline | 489576 | 156 | 228006 | C01879 | 7405 |
| 8 | glucuronic acid | 666401 | 333 | 353273 | | |
| 9 | urea | 325479 | 171 | 296074 | C00086 | 1176 |
| 10 | oxalic acid | 261697 | 147 | 201037 | C00209 | 971 |
| 11 | pseudo uridine | 813744 | 217 | 362150 | | |
| 12 | phosphoric acid | 342472 | 314 | 218342 | C00009 | 1004 |
| 13 | alanine | 243537 | 116 | 199651 | C00041 | 5950 |
| 14 | 2-deoxytetronic | 390395 | 117 | 208880 | | 150929 |
| 15 | serine | 394650 | 204 | 213294 | C00065 | 5951 |
| 16 | histidine | 663393 | 154 | 231328 | C00135 | 6274 |
| 17 | isothreonic acid | 489846 | 292 | 200467 | | 151152 |
| 18 | tyrosine | 671085 | 218 | 199781 | C00082 | 6057 |
| 19 | fructose | 638202 | 307 | 330087 | | 107428 |
| 20 | glutamine | 600028 | 156 | 204386 | C00064 | 5961 |
| 21 | asparagine | 553791 | 116 | 199805 | C00152 | 236 |
| 22 | lysine | 663088 | 156 | 199784 | C00047 | 5962 |
| 23 | uric acid | 731185 | 441 | 221495 | C00366 | 1175 |
| 24 | threonic acid | 497167 | 292 | 199262 | C01620 | 439535 |
| 25 | isoerythronic acid | 494205 | 292 | 308058 | C01620 | 439535 |
| 26 | sorbitol | 667682 | 103 | 204185 | C00794 | 5780 |
| 27 | xylose | 540197 | 103 | 200507 | C00181 | 6027 |
| 28 | 3-aminoisobutyri | 452732 | 248 | 267806 | C05145 | 64956 |
| 29 | lauric acid | 547810 | 117 | 201826 | C02679 | 3893 |
| 30 | glycocyamine | 509822 | 171 | 235721 | C00581 | 763 |
| 31 | glyoxalurea NIS | 480421 | 202 | 339422 | | 4157426 |
| 32 | fucose | 584612 | 117 | 205106 | C01019 | 94270 |
| 33 | glycolic acid | 229810 | 177 | 216047 | C00160 | 757 |
| 34 | stearic acid | 787358 | 117 | 199195 | C01530 | 5281 |
| 35 | levanbiose | 938676 | 217 | 200516 | C01725 | 439555 |
| 36 | arabinose | 546892 | 217 | 202065 | C00259 | 229 |
| 37 | threonine | 409488 | 117 | 321912 | C00188 | 6288 |
| 38 | 2-hydroxyvaleric | 310750 | 131 | 218773 | | 98009 |
| 39 | inulobiose | 930708 | 204 | 202729 | C01711 | 439552 |
| 40 | isocitric acid | 616685 | 245 | 199172 | C00311 | 5318532 |
| 41 | tryptophan | 780702 | 202 | 216485 | C00078 | 6305 |
| 42 | erythritol | 471274 | 205 | 200514 | C00503 | 222285 |
| 43 | gluconic acid | 693140 | 333 | 211990 | C00257 | 10690 |
| 44 | beta-alanine | 431950 | 248 | 227997 | C00099 | 239 |
| 45 | 3-(3-hydroxyphe | 632688 | 267 | 349057 | | 102959 |
| 46 | mannonic acid N | 689431 | 333 | 199322 | C00514 | 3246006 |
| 47 | galacturonic aci | 678170 | 333 | 225841 | C00333 | 84740 |
| 48 | phthalic acid | 567311 | 147 | 213352 | C01606 | 1017 |
| 49 | isothreitol | 464512 | 217 | 308191 | | 169019 |
| 50 | sulfuric acid | 283162 | 227 | 236676 | C00059 | 1118 |
| 51 | arabitol | 572456 | 307 | 362029 | | |
| 52 | xylitol | 566749 | 217 | 200524 | C00379 | 6912 |
| 53 | glyceric acid | 373972 | 189 | 228009 | C00258 | 439194 |
| 54 | indole-3-acetate | 685195 | 202 | 223508 | C00954 | 802 |
| 55 | 2-deoxytetronic | 432896 | 189 | 267848 | | 150929 |
| 56 | leucine | 345953 | 158 | 199607 | C00123 | 6106 |
| 57 | valine | 313224 | 144 | 199605 | C00183 | 6287 |
| 58 | phenol | 217964 | 151 | 200340 | C00146 | 996 |
| 59 | threitol | 467314 | 217 | 202661 | | 169019 |
| 60 | 2-hydroxyglutari | 506359 | 129 | 214409 | C02630 | 43 |
| 61 | pantothenic acid | 691214 | 103 | 205158 | C00864 | 6613 |
| 62 | propane-1,3-diol | 214259 | 177 | 272666 | C02457 | 10442 |
| 63 | ethanolamine | 345242 | 174 | 218991 | C00189 | 700 |
| 64 | N-carbamylgluta | 651580 | 257 | 267745 | C05829 | 121396 |
| 65 | 3-deoxyhexitol N | 618751 | 231 | 374486 | | |
| 66 | 2-deoxyerythritol | 354236 | 117 | 204381 | | 18302 |
| 67 | allantoic acid | 648272 | 100 | 219027 | C00499 | 203 |
| 68 | levoglucosan | 569799 | 204 | 199201 | | 2724705 |
| 69 | rhamnose | 575559 | 117 | 203653 | C00507 | 25310 |
| 70 | phenylalanine | 538016 | 218 | 217642 | C00079 | 6140 |
| 71 | saccharic acid | 699250 | 333 | 202830 | C00818 | 33037 |
| 72 | cysteine | 501345 | 220 | 200918 | C00097 | 594 |
| 73 | 4-hydroxyhippuri | 784510 | 294 | 267712 | C07588 | 10253 |
| 74 | sucrose | 916949 | 271 | 203674 | C00089 | 5988 |
| 75 | pentadecanoic a | 673634 | 117 | 296748 | C16537 | 13849 |
| 76 | UDP-glucuronic | 586393 | 217 | 328006 | C00167 | 17473 |
| 77 | methylhexadeca | 750645 | 117 | 200398 | | 10465 |
| 78 | 2,3-dihydroxybut | 384445 | 292 | 238007 | | 13120900 |
| 79 | benzoic acid | 338043 | 179 | 211970 | C00180 | 243 |
| 80 | glutaric acid | 421227 | 158 | 319882 | C00489 | 743 |
| 81 | syringic acid | 653345 | 179 | 329437 | C10833 | 10742 |
| 82 | 3-deoxypentitol | 527599 | 231 | 267651 | | 12072237 |
| 83 | mannose | 645005 | 205 | 215860 | C00159 | 18950 |
| 83 | glycerol-3-galact | 803554 | 204 | 225851 | C05401 | 656504 |
| 85 | hexuronic acid | 680828 | 333 | 233104 | | 19770757 |
| 86 | 3,6-anhydrogala | 589230 | 231 | 200841 | C06474 | 441040 |
| 87 | 5-hydroxymethyl | 497599 | 123 | 300330 | | 80642 |
| 88 | parabanic acid | 464937 | 100 | 223933 | | 67126 |
| 89 | glucose | 658866 | 160 | 199199 | C00031 | 5793 |
| 90 | dihydro-3-coum | 583527 | 205 | 275091 | C11457 | 91 |
| 91 | 2-aminoadipic a | 573532 | 260 | 203317 | C00956 | 469 |
| 92 | cellobiotol | 956355 | 204 | 200508 | | 160514 |
| 93 | homovanillic + 4 | 601231 | 267 | 322210 | C05582 | 1738 |
| 94 | 4-hydroxymande | 601014 | 267 | 293697 | C05343 | 440639 |
| 95 | N-methylalanine | 286258 | 130 | 205663 | C02721 | 5288725 |
| 96 | aconitic acid | 584220 | 229 | 308113 | C00417 | 309 |
| 97 | indole-3-lactate | 764543 | 202 | 223518 | C02043 | 92904 |
| 98 | glycerol-alpha-p | 591357 | 299 | 199419 | C00093 | 754 |
| 99 | isoleucine | 356726 | 158 | 215089 | C00407 | 6306 |
| 100 | 3,4-dihydroxyhy | 673646 | 179 | 349089 | C10447 | 348154 |
| 101 | methionine sulfo | 637775 | 128 | 218901 | C02989 | 158980 |
| 102 | palmitic acid | 711066 | 313 | 227993 | C00249 | 985 |
| 103 | N-acetyl-D-hexo | 746377 | 103 | 211596 | C03136 | 439281 |
| 104 | cystine | 804143 | 218 | 223490 | C00491 | 67678 |
| 105 | quinic acid | 630216 | 255 | 227967 | C00296 | 6508 |
| 106 | biuret | 571672 | 171 | 228536 | C06555 | 7913 |
| 107 | N-acetyl-D-man | 723859 | 202 | 243111 | C00645 | 65150 |
| 108 | methylhexose N | 616539 | 204 | 238226 | | 8973 |
| 109 | maltose | 946798 | 361 | 204171 | C00208 | 6255 |
| 110 | succinic acid | 370518 | 247 | 199210 | C00042 | 1110 |
| 111 | 2-deoxyribonic a | 544924 | 335 | 232639 | | 22987624 |
| 112 | sophorose | 953453 | 319 | 213152 | C08250 | 441432 |
| 113 | hydroxylamine | 252988 | 146 | 200931 | C00192 | 787 |
| 114 | alanine-alanine | 523055 | 116 | 200449 | | 5484352 |
| 115 | 5-aminovaleric a | 536304 | 174 | 238442 | C00431 | 138 |
| 116 | idonic acid NIST | 698217 | 333 | 199232 | | 193325 |
| 117 | 4-hydroxyphenyl | 541685 | 296 | 308448 | C00642 | 127 |
| 118 | 1-deoxyerythritol | 356844 | 117 | 225848 | | 253154 |
| 119 | sarcosine | 266451 | 116 | 308490 | C00213 | 1088 |
| 120 | 3-hydroxy-3-indo | 778003 | 290 | 267648 | C05635 | 1826 |
| 121 | pelargonic acid | 399114 | 117 | 233458 | C01601 | 8158 |
| 122 | putrescine | 588836 | 174 | 228373 | C00134 | 1045 |
| 123 | 5-hydroxy-3-indo | 777489 | 290 | 348850 | C05635 | 1826 |
| 124 | 4-hydroxyproline | 481319 | 140 | 227980 | C01015 | 825 |
| 125 | 2-ketoglucose di | 456738 | 234 | 201030 | | 159630 |
| 126 | 1,2-anhydro-my | 652000 | 318 | 214408 | | 119054 |
| 127 | proline | 364232 | 142 | 199611 | C00148 | 145742 |
| 128 | furoylglycine NIS | 554295 | 95 | 300332 | | 21863 |
| 129 | ribose | 553606 | 307 | 205473 | C00121 | 5779 |
| 130 | ribitol | 575571 | 217 | 199230 | C00474 | 827 |
| 131 | 1,5-anhydrogluci | 633295 | 217 | 199788 | C07326 | 64960 |
| 132 | noradrenaline | 754834 | 174 | 218832 | C00547 | 439260 |
| 133 | 2,5-furandicarbo | 543827 | 285 | 348921 | | |
| 134 | tartaric acid | 534894 | 102 | 225837 | C00898 | 875 |
| 135 | N-acetylaspartic | 549226 | 274 | 234623 | C01042 | 65065 |
| 136 | ribonic acid | 598019 | 292 | 199341 | C01685 | 5460677 |
| 137 | adipic acid | 473092 | 111 | 308119 | C06104 | 196 |
| 138 | 2-hydroxy-2-met | 264492 | 145 | 300535 | | 95433 |
| 139 | 3-hydroxypropio | 269559 | 177 | 213283 | C01013 | 68152 |
| 140 | gluconic acid lac | 645946 | 220 | 280560 | C00198 | 7027 |
| 141 | xanthine | 702391 | 353 | 203224 | C00385 | 1188 |
| 142 | octadecanol | 755073 | 327 | 199219 | D01924 | 8221 |
| 143 | glycyl proline | 692401 | 174 | 238315 | | 3013625 |
| 144 | galactonic acid | 692399 | 292 | 202883 | C00880 | 128869 |
| 145 | 6-deoxyglucitol N | 595333 | 117 | 200501 | | 151266 |
| 146 | galactinol | 1018547 | 204 | 211957 | C01235 | 439451 |
| 147 | pentitol | 563718 | 217 | 199436 | C00379 | 6912 |
| 148 | phenylethylamin | 512521 | 174 | 272665 | C05332 | 1001 |
| 149 | butane-2,3-diol ( | 204284 | 117 | 225839 | C03044 | 262 |
| 150 | ornithine | 594392 | 174 | 202826 | C00077 | 6262 |
| 151 | diacetone alcoho | 209137 | 115 | 200347 | | 31256 |
| 152 | inositol allo- | 675911 | 318 | 203304 | | 892 |
| 153 | 4-hydroxybenzo | 538420 | 223 | 205107 | C00156 | 105001 |
| 154 | methylcitrate | 628359 | 287 | 267732 | C02225 | 439681 |
| 155 | myristic acid | 634543 | 285 | 199929 | C06424 | 11005 |
| 156 | butyrolactam NI | 277000 | 142 | 200952 | | 12025 |
| 157 | orotic acid | 584723 | 254 | 237885 | C00295 | 967 |
| 158 | cystathionine | 772889 | 128 | 237860 | C02291 | 439258 |
| 159 | fumaric acid | 390708 | 245 | 218753 | C00122 | 444972 |
| 160 | phosphoethanol | 604454 | 100 | 199628 | C00346 | 1015 |
| 161 | pyruvic acid | 208709 | 174 | 362043 | | |
| 162 | arachidic acid | 856486 | 117 | 241206 | C06425 | 10467 |
| 163 | glycero-gulohept | 828907 | 217 | 335217 | | 76599 |
| 164 | 3,4-dihydroxybe | 620248 | 193 | 326579 | C00230 | 77547 |
| 165 | trehalose | 950126 | 191 | 232588 | C01083 | 7427 |
| 166 | N-acetyl-glutami | 603994 | 156 | 362066 | | |
| 167 | citramalic acid | 456337 | 247 | 362044 | | |
| 168 | suberyl glycine | 532293 | 188 | 200526 | | 25202560 |
| 169 | mevalonic acid N | 498738 | 247 | 296075 | C00418 | 439230 |
| 170 | aminomalonic ac | 455266 | 218 | 240264 | C00872 | 100714 |
| 171 | 5-aminovaleric a | 275813 | 170 | 240643 | | 12665 |
| 172 | pipecolic acid | 401074 | 156 | 228067 | C00408 | 439227 |
| 173 | homocystine | 888506 | 128 | 237978 | C01817 | 439579 |
| 174 | 2-deoxyadenosi | 916889 | 192 | 233094 | C00559 | 13730 |
| 175 | 3-hydroxybutano | 278929 | 191 | 199774 | C05984 | 11266 |
| 176 | arginine + ornith | 619420 | 142 | 199796 | C00062 | 232 |
| 177 | glutamic acid | 529370 | 246 | 199604 | C00025 | 33032 |
| 178 | alpha ketoglutari | 507734 | 198 | 200425 | C00026 | 51 |
| 179 | citrulline | 621606 | 157 | 323374 | C00327 | 9750 |
| 180 | glycerol | 344223 | 205 | 199617 | C00116 | 753 |
| 181 | 3-oxopentanedio | 519670 | 257 | 228854 | | 68328 |
| 182 | aspartic acid | 480543 | 232 | 199612 | C00049 | 5960 |
| 183 | capric acid | 451122 | 117 | 213517 | C01571 | 2969 |
| 184 | beta-mannosylgl | 633028 | 259 | 285336 | | |
| 185 | 4-hydroxyphenyl | 653631 | 308 | 308189 | C03672 | 440177 |
| 186 | 1-methyladenosi | 830244 | 259 | 281593 | C02494 | 27476 |
| 187 | erythronic acid la | 437132 | 247 | 308712 | | 5325915 |
| 188 | palatinitol | 1000114 | 319 | 232740 | | 3034828 |
| 189 | lactobionic acid | 952195 | 204 | 214401 | | 517381 |
| 190 | cellobiose | 942646 | 204 | 208839 | C00185 | 294 |
| 191 | (s)-(+)-mandelic | 459578 | 179 | 231101 | | |
| 192 | shikimic acid | 607609 | 204 | 228066 | C00493 | 8742 |
| 193 | paracetamol | 538566 | 206 | 272667 | C06804 | 1983 |
| 194 | 5-methoxytrypta | 863982 | 174 | 200896 | C05659 | 1833 |
| 195 | n-epsilon-trimeth | 511892 | 318 | 269667 | | 440121 |
| 196 | kynurenine | 769271 | 218 | 226885 | C00328 | 161166 |
| 197 | ascorbic acid | 672508 | 332 | 211436 | C00072 | 5785 |
| 198 | 3-methoxytyrosi | 716074 | 218 | 288827 | | 9307 |
| 199 | N-acetyl-D-trypt | 859596 | 202 | 376222 | | |
| 200 | hypoxanthine | 619737 | 265 | 199598 | C00262 | 790 |
| 201 | xylulose NIST | 553050 | 173 | 231908 | C00310 | 5289590 |
| 202 | tagatose | 630162 | 307 | 325185 | D09007 | 92092 |
| 203 | 1-methylinosine | 1027763 | 259 | 234600 | | 65095 |
| 204 | malic acid | 461034 | 233 | 247180 | C00149 | 222656 |
| 205 | 5'-deoxy-5'-meth | 967673 | 236 | 213373 | C00170 | 439176 |
| 206 | O-acetylserine | 415155 | 174 | 268719 | C00979 | 99478 |
| 207 | nonadecanoic a | 822744 | 117 | 241109 | C16535 | 12591 |
| 208 | 3-phosphoglycer | 609618 | 299 | 234616 | C00197 | 724 |
| 209 | 2-hydroxybutano | 258175 | 131 | 199800 | C05984 | 94318 |
| 210 | xanthosine | 924754 | 325 | 237801 | C01762 | 64959 |
| 211 | threose | 446119 | 205 | 349927 | | 439665 |
| 212 | talose | 655071 | 160 | 203596 | C06467 | 99459 |
| 213 | allantoin | 647887 | 331 | 205697 | C01551 | 204 |
| 214 | vanillic acid | 598898 | 297 | 199316 | C06672 | 8468 |
| 215 | dehydroascorba | 633331 | 173 | 200336 | C05422 | 835 |
| 216 | homoserine | 444264 | 218 | 206163 | C00263 | 12647 |
| 217 | xylonic acid | 588743 | 292 | 208695 | | 191545 |
| 218 | 3-ureidopropion | 555429 | 261 | 236805 | C02642 | 111 |
| 219 | N-carbamoylasp | 610736 | 257 | 236701 | C00438 | 93072 |
| 220 | urocanic acid | 700902 | 180 | 268188 | C00785 | 736715 |
| 221 | galactose-6-pho | 817783 | 387 | 199932 | C00446 | 99058 |
| 222 | 1-hexadecanol | 679338 | 299 | 213408 | C00823 | 2682 |
| 223 | 2-hydroxyhippuri | 725431 | 324 | 300349 | C07588 | 10263 |
| 224 | digalacturonic a | 980907 | 169 | 296110 | | 6857565 |
| 225 | melibiose | 981832 | 204 | 200816 | | 11458 |
| 226 | caffeic acid | 748490 | 219 | 268612 | C01481 | 689043 |
| 227 | gentisic acid | 601226 | 355 | 295199 | C00628 | 3469 |
| 228 | glucose-6-phosp | 809066 | 387 | 199350 | C00092 | 5958 |
| 229 | 2-methylglyceric | 372132 | 219 | 228128 | | 152265 |
| 230 | inositol myo- | 725902 | 305 | 228005 | C00137 | 892 |
| 231 | azelaic acid | 610175 | 317 | 225382 | C08261 | 2266 |
| 232 | hydroxycarbama | 325357 | 278 | 203241 | | 18931500 |
| 233 | methylmaleic ac | 418706 | 259 | 202852 | C01732 | 638129 |
| 234 | isopalatinitol | 993209 | 319 | 375098 | | |
| 235 | glucoheptulose | 828610 | 331 | 202881 | | 102926 |
| 236 | cyclohexylamine | 396298 | 200 | 228891 | C00571 | 7965 |
| 237 | benzylalcohol | 281338 | 165 | 199595 | C00556 | 244 |
| 238 | 5-hydroxypiperid | 525088 | 154 | 228012 | | 151730 |
| 239 | 3-phenyllactic ac | 516416 | 193 | 213134 | C05607 | 3848 |
| 240 | enolpyruvate NI | 234457 | 217 | 295001 | C00022 | 1060 |
| 241 | conduritol-beta- | 700238 | 318 | 227948 | | 119054 |
| 242 | uracil | 385903 | 99 | 199600 | C00106 | 1174 |
| 243 | tocopherol alpha | 1067178 | 237 | 199211 | C02477 | 14985 |
| 244 | galactose | 647787 | 160 | 202679 | C00124 | 6036 |
| 245 | 3-hydroxy-3-met | 520721 | 109 | 208716 | C03761 | 1662 |
| 246 | 2-isopropylmalic | 510153 | 275 | 202063 | C02504 | 5280523 |
| 247 | 1,2,4-benzenetri | 521560 | 239 | 294386 | | |
| 248 | phloretin | 945319 | 342 | 202051 | C00774 | 4788 |
| 249 | 3,4-dihydroxyph | 624620 | 384 | 267907 | C01161 | 547 |
| 250 | adenosine | 917818 | 236 | 211944 | C00212 | 60961 |
| 251 | glucito1-6-phosp | 963620 | 387 | 241217 | | 152306 |
| 252 | 5,6-dihydrouracil | 469713 | 243 | 308130 | C00429 | 649 |
| 253 | cyano-L-alanine | 404014 | 141 | 200605 | C02512 | 13538 |
| 254 | chlorogenic acid | 1051689 | 345 | 200513 | C00852 | 1794427 |
| 255 | salicylic acid | 480445 | 267 | 199200 | C00805 | 338 |
| 256 | ferulic acid | 733320 | 338 | 199329 | C01494 | 445858 |
| 257 | thymine | 420134 | 255 | 236696 | C00178 | 1135 |
| 258 | formononetin | 991616 | 340 | 352758 | C00858 | 5280378 |
| 259 | isopalmitic acid | 699712 | 313 | 362085 | | |
| 260 | oleic acid | 779154 | 339 | 215488 | C00712 | 445639 |
| 261 | lactic acid | 216442 | 191 | 199591 | C00186 | 612 |
| 262 | 4-hydroxybutyric | 325197 | 233 | 231100 | C00989 | 10413 |
| 263 | mucic acid | 712231 | 333 | 201866 | C00879 | 3037582 |
| 264 | 2-5-diketopipera | 434367 | 258 | 218596 | C02777 | 7817 |
| 265 | phosphate | 349790 | 314 | 308305 | C00009 | 1004 |
| 266 | guanine | 744497 | 352 | 213138 | C00242 | 764 |

### Example 3. Raw Data Analysis

Pediatric Cohort

326 urine samples were processed for a targeted metabolomics assay that identified 266 metabolites. Sixteen samples had missing data on more than one third of total metabolites identified following a tool called MissForest on non-parametric missing value imputation for mixed-type data. Metabolomics data on the remaining 310 biopsy-matched urine samples was used for the remaining analyses. To evaluate the performance of prediction models, 310 samples were randomly assigned to training (75%) and test (25%) sets. The primary statistical learning method used for allograft outcome prediction was Random Forests via the random Forest package in R. Significant metabolites were selected from the Random Forests model using the VSURF package in R.

The data was normalized against urine creatinine measured as a part of urine metabolome assessment. Non-parametric imputation was applied to these samples via the missForest algorithm. Clustering was performed using and visualized in Morpheus (Broad Institute) using average linkage hierarchical clustering. The log-transformed data was median centered, per metabolite, prior to clustering for better visualization. One minus Pearson's correlation was used for the similarity metric. A fire color scheme was used in heat maps of the metabolites. Z-score analysis scaled each metabolite according to a reference distribution. Unless otherwise specified, the stable phenotype samples were designated as the reference distribution. Thus, the mean and standard deviation of the stable samples were determined for each metabolite. Then each sample, regardless of phenotype, was centered by the stable mean and scaled by the stable standard deviation, per metabolite.

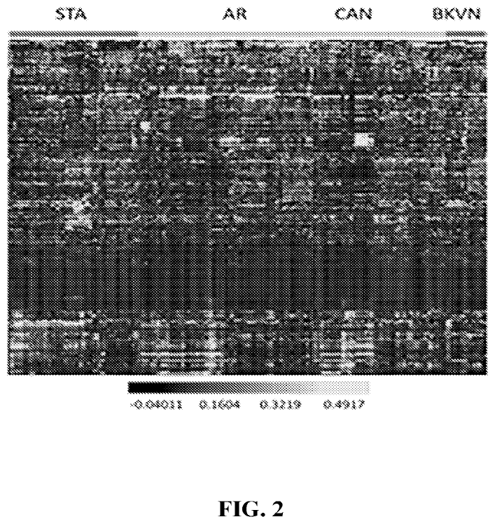

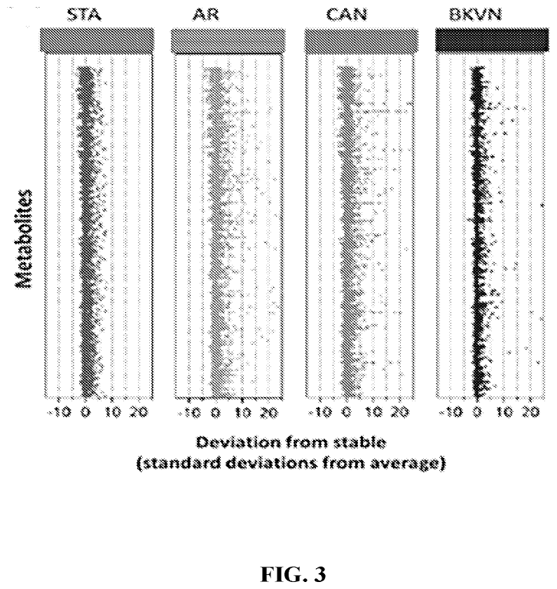

The data was used for supervised clustering to generate a heat map (FIG. 2) and z-score plot (FIG. 3). The heatmap shows heterogeneity in overall metabolome data across urine samples from different phenotypes. In the z-score plot, stable-based z-scores were plotted for each of the 266 metabolites. The plots revealed robust metabolic alterations in AR (z-score range: −4.2 to 800.5) and CAN (z-score range: −3.8 to 265.4) compared to fewer changes in BKVN samples (z-score range: −3.4 to 116.9).

Further methods used for prediction and metabolite selection included LASSO and Elastic Net via the glmnet package in R. Additionally, for visualization of significant metabolites, volcano plots were produced using p-values derived from Welch's t-test with a Bonferroni-adjusted significance threshold of 1.8 e−4 (0.05/266). Comparison of prediction models was done by computing and plotting area under the curve (AUC) from the receiver operating characteristic (ROC) using the pROC package in R. Statistical comparison of AUC values was computed using DeLong's test. Analysis was performed using the R statistical software version 3.4.3.

Adult Cohort:

The Bristol Meyers Squibb (BMS) clinical trial dataset contained 93 patient-matched urine samples—73 in the Belatacept arm and 20 in the Cyclosporin arm (See FIG. 1). 38 of the 93 samples had missing data for at least 1, but less than 10% of all metabolites. Metabolite quantification values were log 2 transformed. To analyze the BENEFIT trial data (belatacept clinical trial originally described by Vincenti, F. et al., N Engl J Med 2016; 374:333-343), a panel of metabolites specific to each treatment arm was produced via VSURF for downstream metabolic pathway analysis (The BENEFIT trial, which analyzed belatacept, was originally described by Vincenti, F. et al., N Engl J Med 2016; 374:333-343).

Because of the low sample size in the Cyclosporin group resulting in high classification error rates, 182 CAN and STA samples treated with calcineurin inhibitors (CNIs) were added increasing the sample size in the Cyclosporin group to 202 for comparison to the 73 Belatacept samples. Metaboanalyst (www.metaboanalyst.ca) was used to perform pathway and molecular networking and interaction analysis. The gene expression data on kidney transplant biopsies was downloaded from NIH/GEO database (https://www.ncbi.nlm.nih.gov/geo/). The datasets used in this analysis were GSE11166, GSE14328, GSE34437, GSE50058, GSE72925, GSE10419, GSE22459, GSE30718, GSE36059, GSE43974, GSE44131, GSE48581, GSE50084, GSE52694, GSE53605, GSE53769, GSE57387, GSE69677, GSE7392, GSE76882, GSE9493, GSE47097, GSE65326. The data was normalized across different datasets. Gene expression differences were calculated using the Wilcoxson rank-sum test. A p value<0.05 was considered significant.

### Example 4. Analysis of Metabolites in Urine Identifies Panels of Metabolites that are Differentially Perturbed in Various Allograft Injuries

Several challenges need to be overcome for an analysis of metabolites in renal allograft recipients to produce significant results. First, the study must be conducted in human renal allograft recipients. Second, the variation seen in sparse panels of metabolites selected for detecting renal allograft injury must be overcome. This is likely due to a number of factors, including differences in sample size, metabolite assay method, and statistical modeling method.

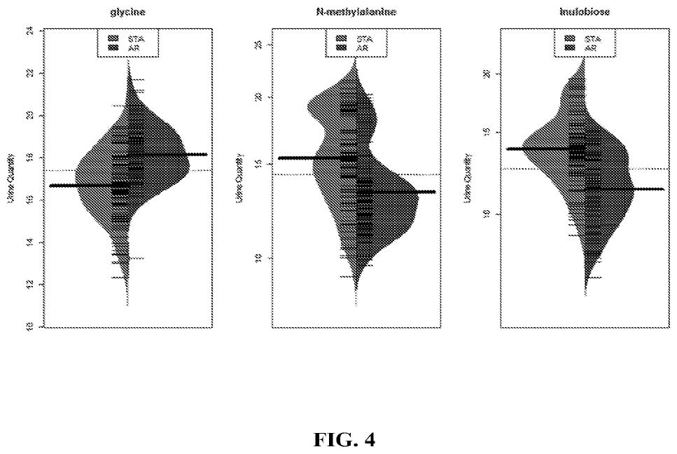

Here, it is demonstrated that a nonlinear, nonparametric machine learning model (i.e. Random Forests) provided a significant improvement over traditional studies in the identification of biomarker panels. Notably, the analysis identified a panel of metabolites that can generally distinguish chronic allograft nephropathy (CAN), acute rejection (AR), BK virus Nephropathy (BKVN) from normal protocol biopsies (STA) using the VSURF method in the Mass Spectrometry (GS-MS) analysis of urine samples. FIG. 4 (FIG. 4) depicts beanplots demonstrating the distribution of the three most significant metabolites in AR compares to STA. The bold horizontal line represents mean value for each group.

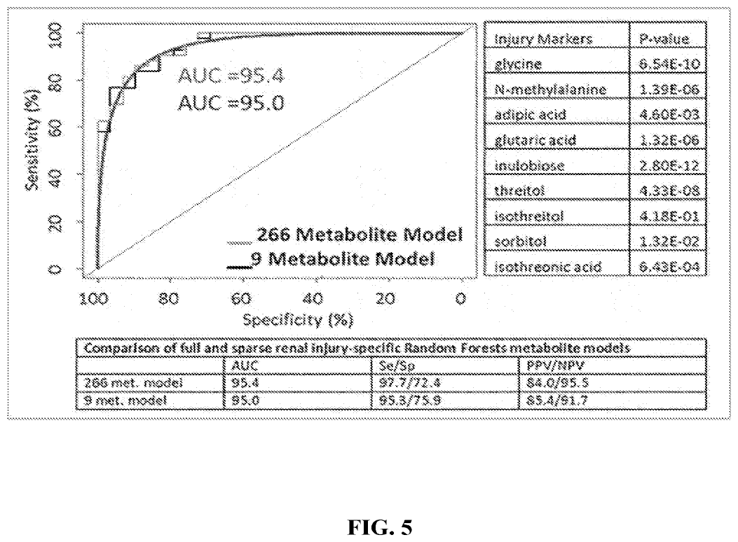

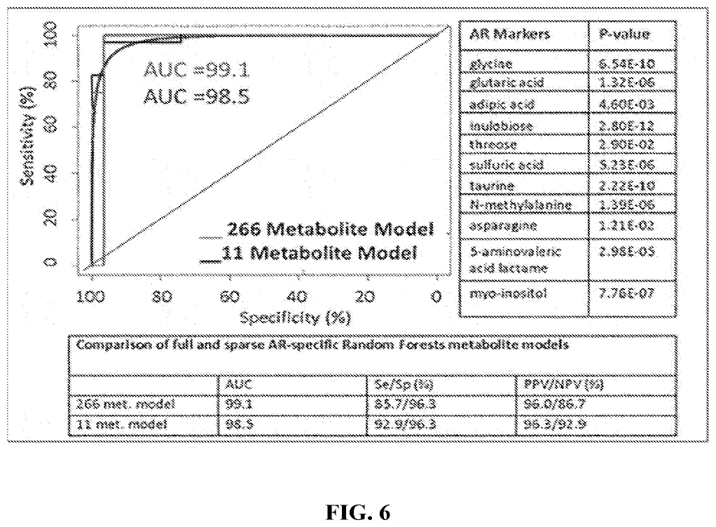

Using VSURF, an AR-specific and an alloimmune injury-specific sparse prediction models performed well on a random test set (98.5 and 95.0 AUC, respectively). Both of these models, using a Random Forests-based variable selection method, produced sparse panels of metabolites out of the total 266 that were deemed important in this prediction (see FIG. 5 and FIG. 6, respectively).

### Example 5. Metabolite Marker Panel for Alloimmune Injury

Applying the VSURF method, a panel of 9 metabolites (FIG. 5) were selected out of 266 to accurately classify post-transplantation alloimmune injury, combining the output from samples with either acute or chronic alloimmune injury (AR/CAN) versus stable (STA) samples. The resulting model had a 95% probability of correctly discriminating between the two outcome groups (AUC=95.0, sensitivity=95.3%, specificity=75.9%).

The 9-metabolite VSURF model was nearly identical in accuracy to the full 266-metabolite model, which had an AUC of 95.4. This difference in AUC values was not significant using DeLong's test (p=0.731), meaning there is no significant change in classification accuracy between the full and abbreviated metabolite models (FIG. 5). This suggests that no diagnostic accuracy is lost in using the abbreviated metabolite model.

TABLE 4
Transplant phenotype specific metabolite markers

| Allograft Injury | AR specific | BKVN specific |
|---|---|---|
| (n = 9) | (n = 11) | (n = 5) |
| glycine | glycine | arabinose |
| N-methylalanine | glutaric acid | 2-hydroxy-2-methylbutanoic |
| | | acid |
| adipic acid | adipic acid | hypoxanthine |
| glutaric acid | inulobiose | benzylalcohol |
| inulobiose | threose | N-acetyl-D-mannosamine |
| threitol | sulfuric acid | |
| isothreitol | taurine | |
| sorbitol | N-methylalanine | |
| isothreonic acid | asparagine | |
| | 5-aminovaleric | |
| | acid lactame | |
| | Myo-inositol | |

### Example 6. Metabolite Marker Panel for Acute Rejection (AR)

In order to identify a metabolite marker panel for acute rejection of kidney transplant VSURF was applied exclusively to the AR and STA urine metabolome datasets (n=217). The resulting model contained metabolites (Table 4, FIG. 6) for AR prediction. The ROC analysis resulted with an AUC of 98.5 with 92.9% sensitivity and 96.3% specificity (FIG. 6). Individual distributions for the three most significant metabolites, glycine, N-methylalanine, and inulobiose, are presented in the form of bean plots (FIG. 4).

### Example 7. BKVN Specific Metabolites

In order to identify BKVN injury specific metabolites, VSURF was used on 22 BKVN urine and 288 non-BKVN urine samples that included AR, CAN, and STA urine. Resulting VSURF panel contained 5 metabolites, Arabinose, 2-hydroxy-2-methylbutanoic acid, hypoxanthine, benzylalcohol, and N-acetyl-D-mannosamine (Table 5) for BKVN prediction with 92.9% sensitivity and 96.9 specificity.

TABLE 5
Predicting BK vs Non-BK from 5 VSURF-selected metabolites

| Prediction | BKVN | AR, STA, CAN | Total |
|---|---|---|---|
| RF(+) | 16 | 6 | 22 |
| RF(−) | 6 | 275 | 281 |
| Total | 22 | 286 | 303 |

Sensitivity = 72.7%;
Specificity = 96.2%;
PPV = 72.7%%;
NPV = 97.9%%;
acc = 96.0%

When the analysis is confined to only BKVN vs STA, the VSURF method identified a panel of 4 metabolites, arabinose, 2-hydroxy-2-methylbutanoic acid, octadecanol, and phosphate to predict BKVN from a pool of samples containing BKVN and STA (Table 6). This subset of predictors improved the prediction accuracy of the model. For this panel, BKVN prediction was 88.9% sensitivity and 94.8% specificity.

TABLE 6
Predicting BK vs. STA from 4 VSURF-selected metabolites

| Prediction | BKVN | STA | Total |
|---|---|---|---|
| RF(+) | 16 | 6 | 22 |
| RF(−) | 2 | 109 | 111 |
| Total | 18 | 115 | 133 |

Sensitivity = 88.9%;
Specificity = 94.8%;
PPV = 72.7%%;
NPV = 98.2%%;
acc = 94.0%

The 4-metabolite VSURF model had accuracy comparable to that of the full 266-metabolite model, which had a sensitivity of 87.5% and specificity of 93.2% (Table 7).

TABLE 7
Predicting BK vs Non-BK from All 266 Metabolites

| Prediction | BKVN | AR, STA, CAN | Total |
|---|---|---|---|
| RF(+) | 13 | 9 | 22 |
| RF(−) | 1 | 277 | 278 |
| Total | 14 | 286 | 300 |

Sensitivity = 92.9%;
Specificity = 96.9%;
PPV = 59.1%;
NPV = 99.6%;
acc = 96.7%

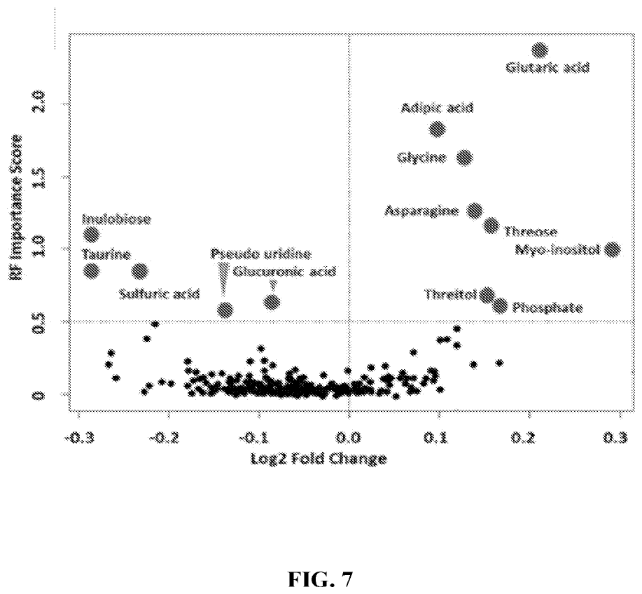

To explore metabolite significance by both statistical significance and magnitude of fold change in the injury group, a volcano plot was generated (FIG. 7). The metabolites with red, brown and purple spots are significantly perturbed with p<1.8 E−4, the metabolites with brown dots are also a listed in 9-metabolite marker panel for alloimmune injury and for AR. The metabolite—taurine with purple dot is listed as a member of 11-metabolite marker panel for AR injury. The plot reveals 32 significant metabolites per the Bonferroni-adjusted threshold of 1.8 e−4 (0.05/266).

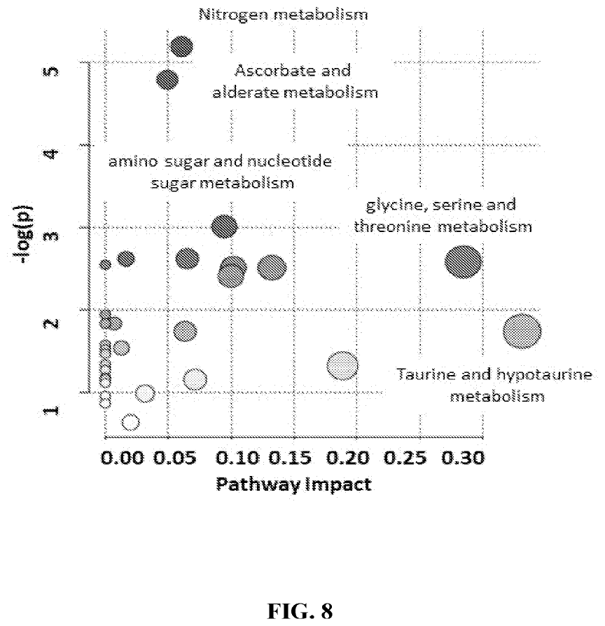

Some metabolites from 9-metabolite marker panel for alloimmune injury and 10-metabolite marker panel for AR are among the very highly perturbed metabolites labeled in brown dots. The metabolites significantly perturbed in kidney transplant injury with p value=0.001 (n=42) were analyzed for metabolic pathway enrichment with Metaboanalyst. Pathway analysis for enrichment identified nitrogen metabolism (p=2.68 E−5), aminoacyl-tRNA biosynthesis (p=0.001) and lysine degradation (p=0.006) were top three significantly enriched pathways (FIG. 8). FIG. 8 (FIG. 8) is a graph illustrating enrichment analysis of metabolic pathways using significantly altered metabolites showed enrichment in nitrogen metabolism (p=0.0055), ascorbate and aldarate metabolism (p=0.0083), and amino sugar and nucleotide sugar metabolism (p=0.05) as significantly enriched pathways.

### Example 8. Analysis of Metabolic Pathways in Belatacept Vs Cyclosporin Treated Subjects

The present disclosure also describes the urine metabolomics approach on samples from a prospective randomized clinical trial of calcineurin inhibitor-based and belatacept-based immunosuppression. As disclosed herein, distinct urinary metabolite pathways were altered in both types of immunosuppression. Overall, targeted metabolomic analyses of biopsy-matched urine samples enabled the generation of refined metabolite panels that non-invasively detect graft injury phenotypes with high confidence.

The metabolic pathways perturbed by immunosuppressive treatment regimens were also evaluated. The samples were collected from subjects administered either a belatacept treatment regimen or a cyclosporin treatment regimen. The VSURF method was applied to identify treatment specific metabolites with the methods described in previous examples, which produced a ranked panel of metabolites for metabolic pathway analysis of both Belatacept and Cyclosporin. Since, no biopsy-matched results were available for these urine samples, Random Forests models were trained on the untreated pediatric cohort and applied to the BENEFIT trial data (n=73) and data from cyclosporine treated subjects (pediatrics and adult combined, n=202). These models were used to classify CAN versus STA samples at 90% classification accuracy, which generated a panel of 135 metabolites for belatacept treated arm and a panel of 83 metabolites for cyclosporine arm. For enrichment of metabolite set enrichment activity and pathways analysis, we used VSURF generated list of metabolites for enrichment and pathway analysis. Table 3 and Table 4 list metabolic pathways specifically enriched in subjects treated with cyclosporine and belatacept in kidney transplantation.

TABLE 8
Cyclosporin specific pathways

| | Metabolic | | | | | Holm | | |
|---|---|---|---|---|---|---|---|---|
| S.No. | pathway | Total | Expected | Hits | Raw p | adjust | FDR | Impact |
| 1 | Glycine, | 48 | 1.0769 | 8 | 7.13E−06 | 0.000571 | 0.000571 | 0.49795 |
| | serine and | | | | | | | |
| | threonine | | | | | | | |
| | metabolism | | | | | | | |
| 2 | Aminoacyl- | 75 | 1.6826 | 8 | 0.0002 | 0.015784 | 0.006302 | 0.05634 |
| | tRNA | | | | | | | |
| | biosynthesis | | | | | | | |
| 3 | Galactose | 41 | 0.91982 | 6 | 0.000236 | 0.018432 | 0.006302 | 0.07793 |
| | metabolism | | | | | | | |

TABLE 9
Belatacept specific pathways

| S. | Metabolic | | | | | Holm | | |
|---|---|---|---|---|---|---|---|---|
| No. | pathway | Total | Expected | Hits | Raw p | adjust | FDR | Impact |
| 1 | Aminoacyl-tRNA | 75 | 2.8355 | 14 | 3.80E−07 | 3.04E−05 | 3.04E−05 | 0.22536 |
| | biosynthesis | | | | | | | |
| 2 | Arginine and | 77 | 2.9111 | 12 | 2.00E−05 | 0.001579 | 0.0008 | 0.37946 |
| | protine | | | | | | | |
| | metabolism | | | | | | | |
| 3 | Nitrogen | 39 | 1.4744 | 8 | 7.19E−05 | 0.005611 | 0.001918 | 0.00067 |
| | metabolism | | | | | | | |
| 4 | Alanine, | 24 | 0.90735 | 6 | 0.000792 | 0.014802 | 0.003845 | 0.65148 |
| | aspartate and | | | | | | | |
| | glutamate | | | | | | | |
| | metabolism | | | | | | | |
| 5 | Glycine, serine | 48 | 1.8147 | 8 | 0.000333 | 0.025332 | 0.005333 | 0.49221 |
| | and threonine | | | | | | | |
| | metabolism | | | | | | | |
| 6 | Glutathione | 38 | 1.4366 | 7 | 0.000424 | 0.031805 | 0.005654 | 0.03761 |
| | metabolism | | | | | | | |
| 7 | Galactose | | | | | | | |
| | metabolism | 41 | 1.5501 | 7 | 0.000689 | 0.050952 | 0.007869 | 0.15399 |

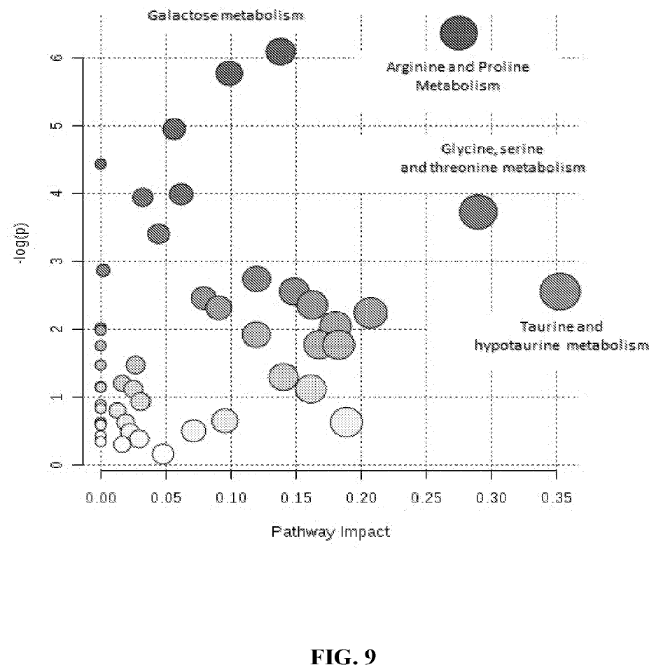

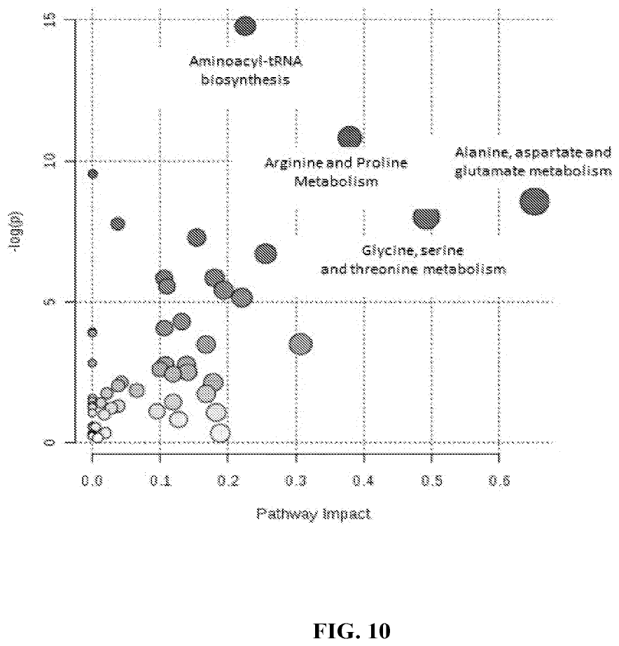

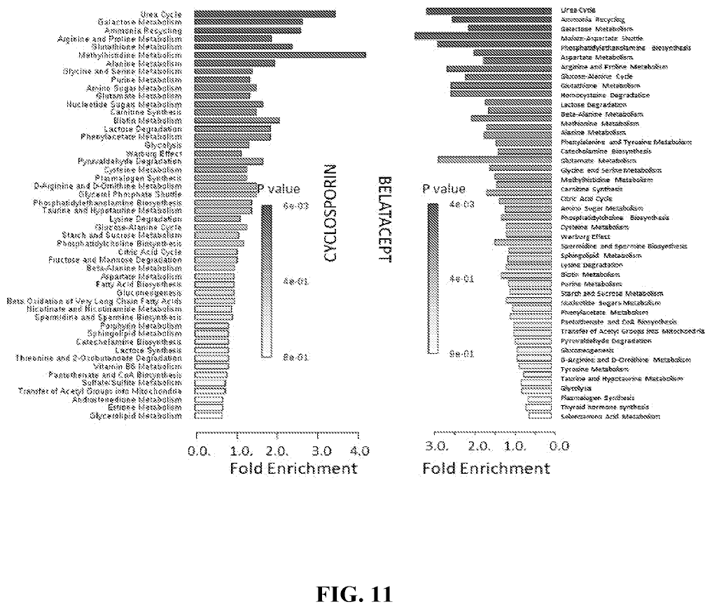

FIG. 9 (FIG. 9) depicts metabolic pathways impacted by Belatacept. FIG. 10 (FIG. 10) depicts metabolic pathways impacted by Cyclosporin. The pathways impacted in cyclosporine, glycine, serine and threonine metabolism (p=′7.13 E−06), aminoacyl-tRNA biosynthesis (p=0.0002), and galactose metabolism (p=0.0002) were also impacted in belatacept treatment arm. In addition, pathways such as arginine and proline metabolism (p=2.00 E−05), nitrogen metabolism (p=′7.19 E−05), and alanine, aspartate and glutamate metabolism (p=0.000192) were significantly perturbed in the belatacept treatment arm (FIG. 10). Two top ranked enriched metabolic sets included galactose metabolism (p=0.0007) and lactose degradation (p=0.01) which are the top ranked enriched pathways for cyclosporine arm which were different from urea cycle (p=0.004) and ammonia recycling (p=0.024) for belatacept treatment arm (FIG. 11). FIG. 11 (FIG. 11) depicts enrichment in metabolic activities based on the urine metabolites specific to each treatment arm.

### Example 9. Validation of Model by Inositol and Myo-Inositol Analysis

We validated some of the VSURF findings by looking into gene expression levels of metabolites that are associated with metabolism or transportation of key metabolites identified in this study. One such metabolite was myo-inositol which was identified as being increased in urine sample of AR subjects.

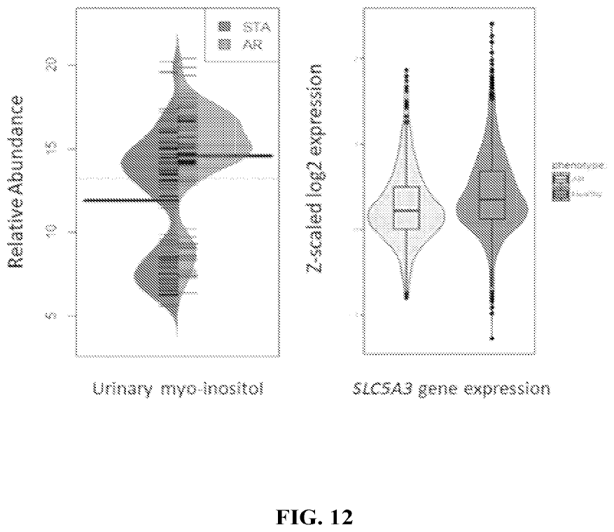

Using gene expression data downloaded from GEO that included 481 kidney biopsies from rejection graft and 1107 from no-rejection graft, we compared gene expression levels of SLC5A3. SLC5A3 was found to be significantly downregulated in the AR group (p=4 e−11) (FIG. 12). This finding is consistent with the manifestation of increased urinary output of myo-inositol. The sodium-myo-inositol transporter (SMIT) located in the proximal tubule, encoded by SLC5A3, functions to reabsorb myo-inositol into the renal medullary cells under condition of hypertonicity. It has recently been suggested that perturbation of this transporter is a mechanism for kidney tissue injury caused by hyperosmolar stress. Our observations support this mechanism as a possible contributor in the progression of acute rejection in transplanted kidneys.

Inositol, specifically myo-inositol, was found to be a significant biomarker in a VSURF model that can discriminate between all four phenotypes tested and was the most important metabolite in discriminating between AR, STA, and CAN. Myo-inositol is an osmolyte of the renal medulla that plays an important role in protecting renal cells from hyperosmotic stress. It is enriched under hyperosmotic conditions via the sodium/myo-inositol cotransporter in the thick ascending limb of the loop of Henle. The kidney is the most important organ for myo-inositol metabolism given that there is high expression of its associated enzymes, L-myo-inositol-1-phosphate synthetase and myo-inositol oxygenase, in the renal parenchyma. Inhibition of myo-inositol transport has been shown to cause acute renal failure in rats. It has recently been shown that, through urine metabolomics profiling of humans, increased levels of myo-inositol is significantly associated with kidney disease and inversely proportional to eGFR. It has also been shown to be elevated in the plasma metabolomic profiles of subjects with end-stage renal disease. The increased level of myo-inositol in urine of subjects with AR can be attributed to decreased gene regulation of transporter of myo-inositol, sodium-myo-inositol transporter (SMIT) located in the proximal tubule, encoded by SLC5A3 gene.

### Example 10. Methods of Treating a Subject Afflicted with Acute Rejection of an Allograft

A subject with end stage kidney disease receives either a deceased-donor (formerly known as cadaveric) or a living-donor kidney in a kidney transplantation. The donor kidney is placed in the lower abdomen and its blood vessels connected to arteries and veins in the recipient's body. When this is complete, blood is allowed to flow through the kidney again. Subsequently, the ureter is connected from the donor kidney to the bladder. In most cases, the kidney will soon start producing urine.

Depending on its quality, the new kidney usually begins functioning immediately. Living donor kidneys normally require 3-5 days to reach normal functioning levels, while cadaveric donations stretch that interval to 7-15 days. Hospital stay is typically for 4-10 days. If complications arise, additional medications (diuretics) may be administered to help the kidney produce urine.

A subject receives a kidney transplant. The subject has proteinuria, an indicator of declining kidney function. The subject that received the kidney allograft provides a urine sample. The urine sample. A mass spectroscopy analysis, either GC-MS, CE-MS, or LC-MS is performed on the urine sample. A panel of metabolites is detected in the urine sample of the subject by the mass spectroscopy.

The results of the mass spectroscopy analysis are evaluated in a nonlinear, nonparametric predictive model of a Machine Learning algorithm, such as the Random Forest model, available in the VSURF package of R. The Random Forest Analysis can be done as follows:

TABLE 10
########### Random Forests Analysis ################
library(“randomForest”, lib loc=“~/R/R-3.4.3/library”)
library(“VSURF”, lib.loc=“~/R/R-3.4.3/library”)
#RF with all variables
RF.UrineMetab.injvno<-randomForest(phenotype '.,
data=train,mtry=17,ntree=500,
na.action=na.omit, importance=TRUE,proximity=TRUE,do.trace=50)
plot(RF.UrineMetab.injvno) #plot of number error by number of trees
varImpPlot(RF.UrineMetab)
View(RF.UrineMetab.injvno$confusion)
#RF 3 outcome
RF.outcome3<-randomForest(outcomemulti '.,
data=train,mtry=80,ntree=1000,
na.action=na.omit, importance=TRUE,
proximity=TRUE,do.trace=50)
plot(RF.outcome3) #plot of number error by number of trees
varImpPlot(RF.outcome3)
View(RF.outcome3$confusion)

The data from the detected panel of metabolites is inputted into a predictive model of a machine learning algorithm, such as the VSURF Random Forest package in R, as described below. The predictive model considers the panel of metabolites and differentiates CAN vs STA vs AR vs BKVN, for example, as illustrated below:

TABLE 11
#RF CAN v STA
RF.outcomeCANSTA<-randomForest(outcomemulti
'.,data=trainCANSTA,mtry=17,ntree=1000, na.action=na.omit,
importance=TRUE,proximity=TRUE,do.trace=50)
plot(RF.outcomeCANSTA) #plot of number error by number of trees
varImpPlot(RF.outcomeCANSTA)
View(RF.outcomeCANSTA$confusion)
RF.CANSTA_pred<-predict(RF.outcomeCANSTA,newdata=testCANSTA)
ROC.RF.CANSTA.pred<-predict(RF.outcomeCANSTA,newdata=testCANSTA, type=‘prob’)
View(table(RF.CANSTA_pred,testCANSTA$outcomemulti))
as.numeric(testCANSTA$outcomemulti)
as.numeric(ROC.RF.CANSTA.pred)
roc(testCANSTA$outcomemulti,ROC.RF.CANSTA.pred[,2])
#RF injvno3 (AR/CAN vs STA)
RF.injvno3<-randomForest(outcomemulti '.,data=injvno3,mtry=80,ntree=1000,
na.action=na.omit, importance=TRUE,proximity=TRUE,do.trace=50)
plot(RF.injvno3) #plot of number error by number of trees
varImpPlot(RF.injvno3)
View(RF.injvno3$confusion)
VSURF.ARSTA<-VSURF(phenotype~.,data=train,ntree=150,mtry=33,na.action=na.omit)
VarSel.RF.ARSTA<-
randomForest(phenotype~me2+me95+me137+me80+me39+me50+me211+me21+me171+me
5,
data=train,mtry=3,ntree=1000, na.action=na.omit,
importance=TRUE,proximity=TRUE,do.trace=50)
VarSel.RF.ARSTA_pred<-predict(RF.ARSTA,newdata=test)
ROC.VarSel.ARSTA.pred<-predict(VarSel.RF.ARSTA,newdata=test, type=‘prob’)
View(table(VarSel.RF.ARSTA_pred,test$phenotype))
#AUC
as.numeric(test$phenotype)
as.numeric(ROC.VarSel.ARSTA.pred)
roc(test$phenotype,ROC.VarSel.ARSTA.pred[,2])
VarSel.RF.injvno3_pred<-as.numeric(VarSel.RF.injvno3_pred)
RF.injvno3_pred<-as.numeric(RF.injvno3_pred)
ROC.injvno3.VSURF<-plot.roc(test$outcomemulti,ROC.VarSel.RF.pred[,2],col=‘blue’,
percent=TRUE, asp=NA,add=FALSE,
main=‘Statistical Comparison of Prediction Models’, lwd=1)
#3 outcome RF model on test set
RF.outcome3_pred<-predict(RF.outcome3,newdata=test)
ROC.RF.pred<-predict(RF.outcome3,newdata=test, type=‘prob’)
View(table(RF.outcome3_pred,test$outcomemulti))
#invno3 RF model on test set
RF.injvno3_pred<-predict(RF.injvno3,newdata=test)
ROC.RF.pred<-predict(RF.injvno3,newdata=test, type=‘prob’)
View(table(RF.injvno3_pred,test$outcomemulti))
#injvno on test set
RF.UrineMetab.injvno_pred<-predict(RF.UrineMetab.injvno,newdata=test)
ROC.RF.pred<-predict(RF.UrineMetab.injvno,newdata=test, type=‘prob’)
View(table(RF.UrineMetab.injvno_pred,test$phenotype))
#VSURF variable selection
VSURF.outcome3<-
VSURF (outcomemulti~.,data=train,ntree=150,mtry=17,na.action=na.omit)
VSURF.outcome3$varselect.pred
#RF with selected variables
VarSel.RF.injvno3<-
randomForest(outcomemultime~2+me95+me137+me80+me39+me59+me49+me236+me41+
me37+me 138+me245,
data=train,mtry=3,ntree-1000, na.action-na.omit,
importance=TRUE,proximity=TRUE,do.trace=50)
plot(VarSel.RF.injvno3) #plot of number error by number of trees
varImpPlot(VarSel.RF.injvno3)
View(VarSel.RF.injvno3$confusion)
#VSURF model on test set
VarSel.RF.injvno3_pred<-predict(VarSel.RF.injvno3,newdata=test)
ROC.VarSel.RF.pred<-predict(VarSel.RF.injvno3,newdata=test, type=‘prob’)
View(table(VarSel.RF.injvno3_pred,test$outcomemulti))

Immunosuppressant drugs are used to suppress the immune system from rejecting the donor kidney. An appropriate treatment is then administered that is tailored to the type of injury afflicting the kidney allograft. These medicines can save subject's life.

The most common medication regimen today for acute rejection includes lymphocyte depleting antibodies, such as an anti-CD20 antibody. The subject may also be treated with one or more of tacrolimus, mycophenolate, prednisolone, ciclosporin, sirolimus, or azathioprine. The risk of early rejection of the transplanted kidney is increased if corticosteroids are avoided or withdrawn after the transplantation.

The subject continues to provide a urine sample over a period of time, for instance, the subject may provide daily urine samples for monitoring if the subject seems to have declining kidney function or proteinuria.

### Example 11. Methods of Treating a Subject Afflicted with Acute Rejection of an Allograft

A subject with end stage kidney disease receives either a deceased-donor (formerly known as cadaveric) or a living-donor kidney in a kidney transplantation. The donor kidney is placed in the lower abdomen and its blood vessels connected to arteries and veins in the recipient's body. When this is complete, blood is allowed to flow through the kidney again. Subsequently, the ureter is connected from the donor kidney to the bladder. In most cases, the kidney will soon start producing urine.

The subject that received the kidney allograft provides a urine sample. A mass spectroscopy analysis, either GC-MS, CE-MS, or LC-MS is performed on the urine sample. A panel of metabolites is detected in the urine sample of the subject by the mass spectroscopy. The results of the mass spectroscopy analysis are evaluated based on predictive models created on support vector machine (SVM) and nonlinear principal manifolds. The analysis was done using 144 metabolites that were selected by support vector machine (SVM) classification with leave one out cross validation (LOOCV). Nonlinear principal manifolds (elastic maps) analysis on the metabolites determined to be significant by Kruskal-Wallis tests was used to delineate stable, AR, IFTA, and BKVN.

Support vector machine and nonlinear principal manifolds analysis was performed as illustrated in Tables 12-15. In the AR symbolic regression model, a score of greater than 0.8623 has a sensitivity and specificity of 92.45% and 91.82% respectively to discriminate between AR and all other outcomes. A score of less than 0.8623 is indicative of stable status.

TABLE 12
AR score (symbolic regression)

| | |
|---|---|
| Area under the ROC curve | |
| Area | 0.9797 |
| Std. Error | 0.006411 |
| 95% confidence interval | 0.9671 to 0.9923 |
| P value | <0.0001 |
| Mann Whitney test | |
| P value | <0.0001 |
| Exact or approximate P value? | Approximate |
| P value summary | **** |
| Significantly different (P < 0.05)? | Yes |

AR_Score = logistic((0.16197862382954*GLUCA*GAP + 0.16197862382954*BF*AD +
0.0444627182906946*CG*Q + 0.16197862382954*CS*min(GAP, GLUCA) −
0.16197862382954*BF*CYSTINE −
0.16197862382954*HYDROXY*DEOXYH)/(ISOCI*MALTO) − 2.88379390390282)
GLUCA = gluconic acid
GAP = glycerol-alpha-phosphate
BF = pipecolic acid
AD = galactonic acid
CG = N-acetyl-D-tryptophan
Q = 1,5-anhydroglucitol
CS = threose
CYSTINE = cystine
HYDROXY = hydroxylamine
DEOXYH = 3-deoxyhexitol NIST
ISOCI = isocitric acid
MALTO = maltose

Table 13—STA Score from symbolic regression. In the STA symbolic regression model, a score of greater than 1.265 e−036 has a sensitivity and specificity of 92.79% and 93.95% respectively to discriminate between STA and all other outcomes. A score of less than 1.265 e−036 is indicative of stable status.

TABLE 13
STA score (SVM)

| | |
|---|---|
| Area under the ROC curve | |
| Area | 0.9728 |
| Std. Error | 0.008227 |
| 95% confidence interval | 0.9567 to 0.9889 |
| P value | <0.0001 |
| Mann Whitney test | |
| P value | <0.0001 |
| Exact or approximate P value? | Approximate |
| P value summary | **** |
| Significantly different (P < 0.05)? | Yes |

STA Score = logistic((50.8238274368658*CV + 50.8238274368658*max(PARA + CELLO,
893.020610883592 + AP + 3.85188369151755*DR))/(CG + DD + EQ +
2.78662417633711*BF) − 93.5757876152196)
CV = vanillic acid
PARA = parabanic acid NIST
CELLO = cellobiotol
AP = butyrolactam NIST
DR = cyclohexylamine
CG = N-acetyl-D-tryptophan
DD = 1-hexadecanol
EQ = lactic acid
BF = pipecolic acid

Table 14—BKVN Score from symbolic regression. In the BKVN symbolic regression model, a score of greater than 9.763 e−011 has a sensitivity and specificity of 95.40% and 85.77% respectively to discriminate between IFTA and all other outcomes. A score of less than 9.763 e−011 is indicative of stable status.

TABLE 14
BKVN score

| | |
|---|---|
| Area under the ROC curve | |
| Area | 0.9948 |
| Std. Error | 0.003163 |
| 95% confidence interval | 0.9886 to 1.000 |
| P value | <0.0001 |
| Mann Whitney test | |
| P value | <0.0001 |
| Exact or approximate P value? | Exact |
| P value summary | **** |
| Significantly different (P < 0.05)? | Yes |

BKVN_Score = logistic((5474.28551967335 + 64.7787180875617*EF +
31.2816315409906*MALTO + 31.2816315409906*EL + 31.2816315409906*ER −
9.68478787083207*ARAB)/AB − 75.4030625057811)
EF = glucitol-6-phosphate NIST
MALTO = maltose
EL = thymine
ER = 4-hydroxybutyric acid
ARAB = arabinose
AB = octadecanol

Table 15—IFTA Score from symbolic regression. In the IFTA symbolic regression model, a score of greater than 9.763 e−011 has a sensitivity and specificity of 95.40% and 85.77% respectively to discriminate between IFTA and all other outcomes. A score of less than 9.763 e−011 is indicative of stable status.

TABLE 15
IFTA score (SVM)

| | |
|---|---|
| Area under the ROC curve | |
| Area | 0.9480 |
| Std. Error | 0.01180 |
| 95% confidence interval | 0.9249 to 0.9712 |
| P value | <0.0001 |
| Mann Whitney test | |
| P value | <0.0001 |
| Exact or approximate P value? | Exact |
| P value summary | **** |
| Significantly different (P < 0.05)? | Yes |

IFTA_Score = logistic(8.65546764045076 + (7.54369709595389*L +
3.23093957093585*ISOCI + 3.23093957093585*M + 3.23093957093585*EQ −
1.36519760238446*THREONINE − 3.23093957093585*INULO − 3.23093957093585*DL −
7.53160154107271*GLUCA − 7.77034923801594*METHYL)/METHYLHEXOSE)
L = 1,2-anhydro-myo-inositol NIST
ISOCI = isocitric acid
M = proline
EQ = lactic acid
THREONINE = threonine
INULO = inulobiose
DL = inositol myo-
GLUCA = gluconic acid
METHYL = methylhexadecanoic acid
METHYLHEXOSE = methylhexose NIST

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of distinguishing a stable kidney allograft from a kidney allograft afflicted by an alloimmune injury comprising:
(a) obtaining a sample from a subject that received a kidney allograft;
(b) detecting a panel of metabolites in the sample; and
(c) distinguishing if the kidney allograft is stable or is afflicted by the alloimmune injury by inputting data from the detection of the panel of metabolites into a predictive model, wherein the output of the model is indicative of allograft status.

2. The method of claim 1, wherein the sample is a urine sample.

3. The method of claim 1, wherein the alloimmune injury is acute rejection.

4. The method of claim 2, wherein the panel of metabolites includes at least one amino acid, at least one amino acid derivative, at least one carbohydrate, and at least one organic compound.

5. The method of claim 2, wherein the panel of metabolites is a 3-metabolite panel.

6. The method of claim 5, wherein the panel of metabolites includes an amino acid, an amino acid derivative, and a carbohydrate.

7. The method of claim 5, wherein the 3-metabolite panel consists of glycine, N-methylalanine, and inulobiose.

8. The method of claim 1, wherein the panel of metabolites is a 11-metabolite panel.

9. The method of claim 8, wherein the 11-metabolite panel has a sensitivity greater than 90% for detecting the acute rejection.

10. The method of claim 8, wherein the 11-metabolite panel has a specificity greater than 90% for detecting the acute rejection.

11. The method of claim 8, wherein the panel distinguishes AR from stable allograft status and the 11-metabolite panel includes glycine, glutaric acid, adipic acid, inulobiose, threose, sulfuric acid, taurine, N-methylalanine, asparagine, 5-aminovaleric acid lactame, and myoinositol.

12. The method of claim 1, wherein the alloimmune injury is chronic allograft nephropathy.

13. The method of claim 12, wherein the panel of metabolites includes a combination of at least one amino acids, at least one amino acid derivative, at least one mineral, at least one carbohydrate, and at least one organic compound.

14. The method of claim 12, wherein the panel of metabolites is a 9-metabolite panel.

15. The method of claim 14, wherein the 9-metabolite panel has a sensitivity greater than 95% for detecting the chronic allograft nephropathy.

16. The method of claim 14, wherein the 9-metabolite panel has a specificity greater than 75% for detecting the chronic allograft nephropathy.

17. The method of claim 14, wherein the 9-metabolite panel includes glycine, N-methylalanine, adipic acid, glutaric acid, inulobiose, threitol, isothreitol, sorbitol, and isothreonic acid.

18. The method of claim 1, wherein the alloimmune injury is BKVN infection.

19. The method of claim 18, wherein the panel of metabolites includes a combination of at least one nucleobase, at least one carbohydrate, at least one fatty acid, and at least one organic compound.

20. The method of claim 18, wherein the panel of metabolites is a 5-metabolite panel.

21. The method of claim 20, wherein the 5-metabolite panel includes arabinose, 2-hydroxy-2-methylbutanoic acid, hypoxanthine, benzylalcohol, and N-acetyl-D-mannosamine.

22. The method of claim 18, wherein the 5-metabolite panel has a sensitivity greater than 85% for detecting the BKVN infection.

23. The method of claim 18, wherein the 5-metabolite panel has a specificity greater than 90% for detecting the BKVN infection.

24. The method of claim 18, wherein the panel of metabolites is a 4-metabolite panel.

25. The method of claim 24, wherein the 4-metabolite panel includes arabinose, 2-hydroxy-2-methylbutanoic acid, octadecanol, and phosphate.

26. The method of claim 24, wherein the 4-metabolite panel has a sensitivity greater than 85% for distinguishing the BKVN infection from a stable allograft.

27. The method of claim 24, wherein the 4-metabolite panel has a specificity greater than 90% for distinguishing the BKVN infection from a stable allograft.

28. The method of claim 1, wherein the panel of metabolites is detected by a mass spectroscopy analysis.

29. The method of claim 28, wherein the mass spectroscopy analysis is a gas chromatography-mass spectrometry (GC-MS) analysis.

30. The method of claim 28, wherein the mass spectroscopy analysis is a capillary electrophoresis-mass spectrometry (CE-MS) analysis.

31. The method of claim 28, wherein the mass spectroscopy analysis is a liquid chromatography-mass spectrometry (LC-MS) analysis.

32. The method of claim 1, wherein the predictive model is a supervised learning model that has been trained on a biopsy matched cohort of samples.

33.-57. (canceled)
